(19) United States
(12) Patent Application Publication
(10) Pub. No.: US 2008/0206753 A1
(43) Pub. Date: Aug. 28, 2008

(54) Methods for Cancer Prognosis

(75) Inventors: Sean E. Egan, Toronto (CA); Michael Reedijk, Toronto (CA)

Correspondence Address: MYERS BIGEL SIBLEY & SAJOVEC, PO BOX 37428, RALEIGH, NC, 27627, US

(73) Assignee: THE HOSPITAL FOR SICK CHILDREN, TORONTO ONTARIO (CA)

(21) Appl. No.: 11/817,770

(22) PCT Filed: Mar. 6, 2006
(86) PCT No.: PCT/CA06/00318

§ 371 Date: Apr. 18, 2008

Related U.S. Application Data
(60) Provisional application No. 60/658,189, filed on Mar. 4, 2005.

(51) Int. Cl. C12Q 1/68; A61P 35/00

(52) U.S. Cl. 435/6

(57) ABSTRACT

A method for assessing prognosis in a subject having a breast tumor comprises determining the level of expression of at least one Notch receptor gene, Notch ligand gene or Notch signaling target gene. A method of treating a subject suffering from a breast tumor associated with increased Notch signaling comprises administering to the subject an effective amount of an inhibitor of Notch signaling.

### FIELD OF THE INVENTION

The invention relates to methods for diagnosing or assessing prognosis of breast tumors. It further relates to methods and compositions for treating breast tumors and to methods for screening potential therapeutic compounds.

### BACKGROUND OF THE INVENTION

Breast cancer is the most commonly diagnosed malignancy, and a leading cause of cancer death, in western females (1). A continued focus on elucidating molecular mechanisms underlying this disease is necessary to. improve on current limited treatment success. Mutations in human breast cancer have been identified that activate expression or elevate function of oncogenes and that disrupt tumor suppressor genes. In some cases, specific molecular lesions have been associated with poor prognosis (2, 3) or with specific breast tumor types (4). In addition, microarray studies have helped to define gene expression profiles that correlate with patient outcome (5). Insight into mechanisms whereby breast tumors grow and evolve has also come from the study of hormone or growth factor systems that control normal mammary epithelial cell proliferation, differentiation, and survival. For example, steroid hormones play a major role in regulating normal and malignant mammary cell biology. One of the most important therapeutic strategies in the treatment of human breast cancer is targeted towards disrupting estrogen receptor function. Indeed, learning how normal mammary gland growth and differentiation are regulated may illuminate a path to improved treatment for breast cancer.

Studies on mouse mammary tumor virus (MMTV)-induced breast cancer have led to the identification of several developmental regulatory genes with potential to control mammary epithelial cell division, differentiation and survival (6). For example, Wnt genes, which are now known to regulate normal development of most tissues including the mammary gland, were discovered in this system (7). Several Fibroblast Growth Factor (FGF) genes were also discovered as MMTV-activated oncogenes, and FGFs are now known to regulate mammary biology at multiple levels (8, 9). The third developmental gene family implicated in this mouse breast cancer model system is the Notch receptor family (10).

Notch receptors are large transmembrane EGF-like repeat-containing proteins that regulate many cellular properties, including cell division, differentiation, sorting, migration, fate specification, morphology, and survival (11-13). Mammals have four Notch receptors: Notch1, 2, 3, and 4. These receptors are activated in most contexts by mammalian Delta (DII) and Serrate (Jagged) ligands, which are also transmembrane proteins containing multiple EGF-like repeats (known as DSL ligands). In addition, the specificity of Notch receptors for these ligands is regulated by Fringe-family sugar transferase enzymes, which extend O-linked fucose residues on both receptor and ligand through addition of GlcNAc (14, 15).

Once activated, Notch receptors are cleaved to release a cytoplasmic domain fragment that translocates into the nucleus where it converts a transcriptional repressor complex into a transcriptional activation complex (16, 17). This complex is nucleated by RBPJκ/CBF-1, a conserved DNA protein which controls expression of many genes involved in cell growth and differentiation. Notch receptors can also directly activate signal transduction pathways in the cytoplasm, including pathways involving Deltex (18), AbI tyrosine kinases(19), NFκB(20), Disheveled (21), STAT3 (22), Smad (23), and PI3K/Aκt (24, 25).

Notch signaling has been implicated in the development of organs and tissues derived from all three germ layers. Interestingly, Notch signaling plays an important role in development of skin, blood vessels and fat. Mammary epithelium is a specialized derivative of the skin that develops coordinately with mammary vessels and adipose stroma (26-28).

Work by Callahan and colleagues showed that an activated Notch4 could transform mammary epithelium in vitro (10) and in vivo (29-32). Interestingly, an activated Notch4 oncogene (Int3) slowed ductal growth and perturbed lobular outgrowth prior to induction of tumor formation (30). Activated Notch4 had the opposite effect to Wnt signaling on TAC-2 mammary epithelial cell branching in vitro (33), suggesting that these two pathways transform cells through very distinct mechanisms. Activated Notch1 can also transform mammary epithelium (34, 35).

As noted above, hyperactivation of Notch signaling alters mammary development and ultimately promotes mammary epithelial transformation (30, 31).

Parr et al. (36) have shown that Notch1 mRNA levels were statistically higher in high-grade tumors compared to low-grade tumors. The authors of this study were, however, unable to demonstrate any correlation between high levels of Notch1 expression and poor-outcome. No study to date has conclusively related the level of Notch 1 or 3 receptor expression and outcome in human breast cancer.

There remains a need for tests which enable the prediction of likely outcome in breast cancer patients, to assist in the selection of appropriate therapies.

### SUMMARY OF THE INVENTION

The invention provides a method for assessing the prognosis for a subject having a breast tumor by determining the level of expression of a Notch receptor gene, a Notch ligand gene and/or a Notch signaling target gene. Increased expression of the gene indicates a poorer prognosis.

The subject may be a human subject.

The invention further provides a method for assessing the prognosis for a subject having a breast tumor, comprising determining the level of expression of at least one gene selected from the group consisting of Notch 1, Notch 3, Jagged1, Hey 1, Hev 2, HevL and Hes 1 to Hes 7 in a sample of the tumor, wherein the higher the level of expression of the at least one gene in the tumor, the poorer the prognosis for the subject.

In a further embodiment, the expression of at least one of Notch 1, Notch 3 and Jagged 1 is determined, and in further embodiments, the expression of at least two or of all three of these genes is determined.

In a further embodiment, the subject is a human subject and the level of expression of at least one of NOTCH 1, NOTCH 3 and JAG1 is determined.

In a further embodiment, a method is provided for diagnosing breast cancer in a subject comprising determining the level of expression of at least one Notch receptor gene, Notch ligand gene or Notch signaling target gene in a breast tissue sample obtained from the subject.

In a further embodiment, a method is provided for diagnosing breast cancer in a subject comprising determining the level of expression of at least one gene selected from the group consisting of Notch 1, Notch 3, Jagged 1, Hey 1, Hey 2, HevL, Hes 1, Hes 2, Hes 3, Hes 4, Hes 5, Hes 6 and Hes 7 in a breast tissue sample obtained from the subject, wherein an increased level. of expression of the at least one gene compared to the level of expression of the at least one gene in normal breast tissue is indicative of breast cancer in the subject.

In a further embodiment, a method is provided for treating a subject suffering from a breast tumor associated with increased Notch signaling comprising administering to the subject an effective amount of an inhibitor of Notch signaling.

In a further embodiment, a method is provided for treating a subject suffering from breast cancer by administering to the subject a pharmaceutical compound which reduces the expression of at least one of Notch 1, Notch 3, Jagged 1, Hey 1, Hey 2, HeyL and Hes 1 to Hes 7 or reduces activity of at least one of Notch 1, Notch 3, Jagged 1, Hey 1, Hey 2, HeyL and Hes 1 to Hes 7.

Such pharmaceutical compounds include γ-secretase inhibitors.

In a further embodiment, a method is provided for screening a candidate compound for its potential usefulness in the treatment of breast cancer comprising contacting a tumor cell or cells with the candidate compound under conditions which permit expression of at least one gene selected from the group consisting of Notch 1, Notch 3, Jagged 1, Hey 1, Hey 2, HeyL and Hes 1 to Hes 7 and determining the level of expression of the at least one gene in the tumor cell or cells, wherein a lower level of expression compared to the level of expression in the cell or cells in the absence of the compound indicates the potential usefulness of the compound in the treatment of cancer.

### SUMMARY OF THE DRAWINGS

Certain embodiments of the invention are described, reference being made to the accompanying drawing, wherein:

FIG. 1 shows Kaplan-Meier curves of the relationship between overall survival (Y axis) and months of follow up (X axis) in breast cancer patients having tumors expressing high (broken line) or low (solid line) levels of JAG 1 (FIG. 1a), NOTCH 1 (FIG. 1b), or NOTCH 3,(FIG. 1c); or co-expressing high JAG 1 and NOTCH 1 (broken line) or all others (FIG. 1d); or high JAG 1 and NOTCH 1 (broken line), high JAG 1 (dotted line), high NOTCH 1 (top line) and neither high JAG 1 nor NOTCH 1 (solid line) (FIG. 1e).

FIG. 2A shows overall survival (X axis) and months of follow up (Y axis) in patients having breast tumors expressing high (broken line) or low (solid line) levels of JAG 1.

FIG. 2B shows overall survival (X axis) and months of follow up (Y axis in patients having breast tumors expressing high (broken line) or low (solid line) levels of NOTCH 1.

### DETAILED DESCRIPTION OF THE INVENTION

It is desirable, before embarking on invasive and debilitating treatment for cancer, to try to identify patients most likely to have a poor outcome, who are therefore candidates for the most vigorous therapy.

The present invention provide new methods for diagnosing breast cancer, for assessing the prognosis for a breast cancer patient, for predicting the susceptibility of a tumor to agents that interfere with expression of Notch ligands, receptors, fringes or signaling through the Notch pathway, and for treating breast cancer patients with an inhibitor of Notch signaling.

Notch ligands, receptors and signaling targets are referred to herein by their generic designations, eg. Notch1, Notch3, Jagged1 or by their species-specific designations, eg. for humans, NOTCH1, NOTCH3, JAG1. Where Notch ligands, receptors and signaling targets are referred to by their generic designations, in the context of a particular species, the species-specific Notch ligand, receptor or signaling target is inferred.

It has been found that the genes for one or more of NOTCH1, NOTCH 3, and JAG 1 are highly expressed in a subset of human breast tumors and that this subset of tumors comprises those with most pathological features indicative of a poor prognosis. The pathological features studied are discussed in Example 2.

Statistical analysis has shown, as described in the examples herein, that patients whose breast tumors showed a higher level of expression of these genes, compared with the expression level in normal breast tissue, had a poorer prognosis than patients whose breast tumors did not show such higher expression levels. Additionally, the greater the increase in expression level over normal, the poorer the prognosis for the patient, meaning the poorer the clinical outcome, as indicated by progressive disease and ultimately death. The aggressiveness of therapy desirable may be indicated by the prognosis, as indicated by the increase in expression level of the selected genes.

The data described herein provide the first direct evidence for a relationship between high-level JAG 1 and/or NOTCH 1 expression and poor overall patient survival in human breast cancer. Without wishing to be bound by this theory, these studies suggest that a JAG 1-NOTCH 1 activation loop is functioning to promote tumor formation and progression in the same way that MMTV-activated Notch1 does in mice. Consistent with this, Pece et al. have identified a group of Numb-negative human breast tumors where Notch signaling appears to be activated, at least when cells are cultured ex vivo (44). The potential for JAG1-mediated autocrine or juxtacrine Notch signaling in cancer has been established in a number of systems.

The Hes genes and Hey genes are well-defined targets of Notch signaling, as discussed for example in Callahan et al. (13). Stylianou et al. (57) have recently observed that aberrant activation of Notch turns on Hey gene expression in human breast cancer.

It is therefore likely that increased expression of one or more of the Hes or Hey genes will also be indicative of poor prognosis in breast tumor patients.

In one embodiment of the invention, a method for assessing prognosis for a subject having a breast tumor comprises determining the level of expression of at least one Notch receptor gene, Notch ligand gene or Notch signaling target gene in the tumor.

A Notch signaling target gene or Notch target gene is a gene whose expression is regulated by Notch signaling. Such genes include the Hey and Hes genes, including Hey 1, Hey 2. HevL, Hes 1, Hes 2, Hes 3, Hes 4, Hes 5, Hes 6 and Hes 7.

In a further embodiment, a method for assessing the prognosis for a subject having breast tumor, comprises determining the level of expression of at least one gene selected from the group consisting of Notch 1, Notch 3, Jagged 1, Hey 1, Hey 2, HevL and Hes 1 to Hes 7.

In a further embodiment, the expression levels of at least one of NOTCH 1, NOTCH 3, and JAG 1 is determined.

In a further embodiment, the expression level of JAG 1 and NOTCH 1 are determined.

In a further embodiment, the expression level of one or both of HEY1 and HES5 is determined.

Determination of an increased level of expression of at least one gene from the group described herein includes determination of expression in a tissue where no expression is detectable in the corresponding normal tissue and also determination of a higher level of expression than the level observed in the corresponding normal tissue.

Determination of the level of expression of at least one gene in a breast tumor sample, as described herein, can serve to assist a clinician in determining an appropriate approach to management of the tumor patient. The higher the increase in expression over the level of expression in normal tissue, the greater the need for aggressive treatment. For example, a level of expression which is two fold higher than the level of expression in normal tissue, for example five fold higher, or as further example ten fold higher, is indicative that more aggressive therapy is desirable.

As will be appreciated by those of skill in the art, the method of determining prognosis based on increased gene expression, as described herein, may also be used after therapy to detect a recurrence of an aggressive breast tumor.

In a further embodiment, the invention provides a method of diagnosing breast cancer in a subject comprising determining the level of expression of at least one of Notch 1, Notch 3, Jagged 1, Hey 1, Hey 2, HevL, Hes 1, Hes 2, Hes 3, Hes 4, Hes 5, Hes 6 and Hes 7 in a breast tissue sample obtained from the subject.

Breast tissue samples are routinely obtained by biopsy for examination as part of the process of diagnosing breast cancer in a subject. Such samples may be examined by the methods described herein for an increased level of expression of at least one of the genes described in the preceding paragraph, relative to the expression level of the at least one gene in normal breast tissue, either from the same subject or a normal breast tissue reference sample. An increased level of expression in the biopsy sample is suggestive of breast cancer in the subject.

Determining the expression level of a gene in a tumor means determining the level of RNA transcripts or the level of protein expression in the tissue.

Determination of the expression level of one or more of Notch 1, Notch 3, Jagged 1, Hev 1, Hey 2, HeyL and Hes 1 to Hes 7 may be carried out using nucleic acid-based tests or tests based on the expressed protein.

Tissue samples for testing are obtained from breast tissue or tumor biopsy carried out by standard surgical techniques. The subject may be a human or an non-human animal, such as a non-human primate, cat, dog, cow, horse, sheep or pig.

Human breast tissue samples, for example 0.6 mm sections, have been found suitable. Smaller samples such as micro dissected samples could also be used, employing highly sensitive assays which are well known to those skilled in the art, for example PCR/micro array or quantitative PCR-based assays or an approach using protein-DNA chimeras as described in Burbulis et al. (58).

Suitable methods for determining the expression level of a gene, whether based on mRNA or protein product, are well known to those of skill in the art. Examples are described herein and further suitable protocols can be found, for example, in Ausubul et al. (59).

Nucleic acid-based tests for determining expression of Notch 1, Notch 3, Jagged 1, Hey 1, Hey 2, HeyL and Hes 1 to Hes 7 include hybridization assays to assess tissue mRNA levels in tissue sections as described herein. Selection of suitable specific probes for a particular Notch ligand, Notch signaling target or Notch receptor gene of the species on which the test is to be carried out is within the skill of those in the art. Suitable probes include radiolabelled fragments of a cDNA encoding Notch 1, Notch 3, Jagged 1, Hey 1, Hey 2, HeyL and Hes 1 to Hes 7. Such cDNAs may be obtained, for example, from the IMAGE consortium. The cDNA sequences of these genes are available through the Genbank DNA sequence database, housed at the National Center for Biotechnology Information (http://www.ncbi.nlm.nih.gov/). Alternatively, these sequences can be obtained from original journal articles (37 to 39).

A specific oligonucleotide probe of at least about 17 nucleotides may be employed. Probes may be at least about 100 nucleotides, or at least about 200 nucleotides or at least one or more kilobases.

Notch signaling target, ligand and receptor gene expression may also be determined using extracted mRNA using microarray based technology. For example, one can hybridize in vitro fluorescently-labeled tumor cRNA to probe a small tailor-made chip with probes for JAG 1, NOTCH 1, NOTCH 3, as well as various reference genes, to determine absolute (normalized) levels of expression for each gene. The chip is also designed with additional controls to determine how much Notch ligand, target or receptor gene expression was coming from tumor vs. non-tumor tissue. For example, the chip includes reference standards for blood vessel gene expression and for expression of other common stromal cell types. In this way, the tumor-specific expression of a Notch ligand, receptor, Fringe or Notch target gene (such as Hes or Hey genes) could be calibrated through subtraction of the normalized stromal expression level for each gene. An algorithm can be established, based on the expression of vessel specific genes or other stromal specific genes, to determine the absolute value of Notch, ligand, receptor, Fringe, or Notch target gene expression to be subtracted. As an example, JAG1 is expressed at level A^{Norm} on the microarray (after normalization using the housekeeping reference gene standards). At the same time a vascular marker gene is expressed at level Y^{vesmark} in this sample. Based on in situ hybridization experiments one identifies a series of tumors that do not express JAG1, but that contain varying densities of blood vessels. By comparing normalized microarray expression values of JAG1 (A^{Norm}) for these tumors to normalized expression levels for vessel reference gene(s) (Y^{vesmark}), a multiplication value is generated to convert vessel marker gene expression values into vessel-specific JAG1 expression values for each sample (V vessel JAG1 factor) (note that JAG1 is expressed in blood vessels). Therefore, to generate the normalized tumor-specific JAG1 expression value (JAG1^{TuExNorm}), the formula; JAG1^{TuExNorm}=A^{Norm}−Y^{vesmark}×V. is used. The tailor made microarray would optimally also contain probes to establish normalized expression of all Notch ligands, receptors, Fringes, and Notch target genes, as well as other prognostic indicators such as HE/Neu/erbB2.

In a further embodiment, the test may be based on the proteins expressed from one or more of the Notch receptor or Notch target or ligand genes discussed above, using methods such as western blots or immuno-histochemical analysis using antibodies specific for the protein(s) or fragments thereof on tissue sections in conventional methods known to those of skill in the art. Antibodies to these proteins or fragments thereof may be prepared by conventional methods.

If a tissue sample is large enough to contain both tumor tissue and normal tissue, eg. non-tumor mammary epithelium, the expression level of the selected Notch receptor or Notch ligand or target gene or genes may be determined in the subject's own normal tissue and compared to the expression level in the tumor tissue. The higher the expression level found in the tumor tissue, compared to the normal tissue, the poorer the prognosis for the tumor-bearing subject and the greater the need for aggressive anti-tumor treatment.

In the tumor tissue may be compared to the level of expression in a normal breast tissue reference sample or may be compared to the level of expression of a series of reference genes, such as housekeeping genes, as reported in micro-array studies of gene expression in large numbers of tumors, for example large numbers of mammary tumors (50 to 52).

The invention further provides a method for treating a subject suffering from a breast tumor associated with increased Notch signaling comprising administering to the subject an effective amount of an inhibitor of Notch signaling. A tumor associated with increased Notch signaling can be identified by determining the level of expression of a Notch receptor-, ligand or signaling target gene such as Notch 1, Notch 3, Jagged 1, Hey 1, Hey 2, HeyL and Hes 1 to Hes 7 as described herein.

Inhibitors of Notch signaling include compounds which inhibit the expression of one or more genes in the Notch signaling pathway and compounds which inhibit or reduce the activity of the protein product of such expression.

As used herein, “an inhibitor of Notch signaling” includes a compound which inhibits Notch signaling and a mixture of more than one compound, each of which inhibits Notch signaling.

The invention further provides methods for treating patients suffering from breast cancer, by administering a pharmaceutical compound or composition which reduces expression of at least one of Notch 1, Notch 3 and Jagged 1, Hey 1, Hey 2. HeyL and Hes 1 to Hes 7 or reduces the activity of their expressed proteins. For example, antisense oligonucleotides or siRNA species which hybridise to the DNA of one of these genes or to a corresponding mRNA, and prevent transcription or translation, so that production of the encoded protein is reduced or prevented, may be employed (53-55).

In a further embodiment, a compound or composition which inhibits the activity of the at least one protein expressed from any of Notch 1, Notch 3, Jagged 1, Hey 1, Hev 2, HevL and Hes 1 to Hes 7 may be employed. For example, Kuzbanian/TACE protease inhibitors or γ-secretase inhibitors could be used to block Notch activation and/or signaling, since these proteases are required to activate Notch signaling (40), and gamma-secretase inhibitors have been shown to display anti-Notch activity (49).

Many γ-secretase inhibitors have been described and some of these are in an advanced stage of development as pharmaceuticals in clinical trials for the treatment of Alzheimer's disease (40 to 43).

In a further embodiment of the invention, a method is provided for screening a candidate compound for its potential usefulness in the treatment of breast cancer comprising contacting a tumor cell or cells with the candidate compound under conditions which permit expression of at least one gene selected from the group consisting of Notch 1, Notch 3, Jagged 1, Hey 1, Hey 2. HevL and Hes 1 to Hes 7 and determining the level of expression of said at least one gene in the tumor cell or cells, wherein a lower level of expression compared to the level of expression in the cell or cells in the absence of the compound indicates the potential usefulness of the compound in the treatment of cancer.

Conventional methods for determining the level of expression of a gene may be employed, such methods being known to those of skill in the art. Such methods include the methods described herein and the method described by Burbulis et al. (58).

### EXAMPLES

The examples are described for the purposes of illustration and are not intended to limit the scope of the invention. Methods of chemistry, molecular biology, protein and peptide biochemistry and histology referred to but not explicitly described in this disclosure and examples are reported in the scientific literature and are well known to those skilled in the art.

### Material and Methods

Breast tissue paraffin blocks. Human breast cancer specimens were obtained through a written informed consent process that adheres to stringent ethical criteria at the University Health Network of the University of Toronto. Within 5-15 minutes of resection, tumor specimens were placed in an RNAse-free solution of 4% paraformaldehyde (PFA) in phosphate-buffered saline pH 7.4 (PBS) and incubated at room temperature (RT) overnight to allow fixation. The tissues were then washed for 30 minutes in each of PBS, saline, saline:ethanol (1:1) and 70% ethanol in saline. Paraffin blocks were generated in the standard fashion.

Slide preparation for in situ hybridization. 6 μm microtome sections of paraffin-embedded tumor tissue were cut and placed onto glass slides. Slides were warmed to 42° C. for 30 minutes, and then incubated with dessicant at RT overnight (ON).

Breast Cancer Tissue Micro-arrays. Breast cancer tissue micro-array (TMA) slides were provided by the Cooperative Breast Cancer Tissue Resource, which is funded by the National Cancer Institute (NCI), U.S. These TMAs included one hundred and ninety two 0.6 mm samples of invasive primary ductal breast cancer (64 cases each of node-negative, node-positive and metastatic breast cancer).

Generation of probes for in situ hybridization. cDNAs encoding Notch receptors, ligands, and Fringes were obtained from the IMAGE consortium. Portions of these were subcloned into expression vectors using standard techniques. To generate radio-labeled probes for in situ hybridization, plasmid DNA was linearized, and then desalted and concentrated using a Qiaex II gel extraction kit. Linearized DNA was incubated with 0.5 mM nucleotides (ATP, GTP, CTP), RNAse inhibitor (0.5 U/μl; Invitrogen), ³³P-UTP (2.5 uCi/μl; Amersham) and either T7 or T3 polymerase (0.5 U/μl; Roche) in polymerase buffer (40 mM Tris-HCl pH 8.0, 6 mM MgCl₂, 10 mM dithiothreitol [DTT], 10 mM spermidine) at 37° C. for 30 minutes. Additional polymerase was added and the reaction allowed to proceed for a further 45 minutes. Next, plasmid DNA was digested using RNAse-free DNAse 1 (4 U/μl; Invitrogen) leaving intact sense or antisense radio-labeled RNA probe. Probes were purified using ProbeQuant™ G-50 micro columns (Amersham).

In situ hydridization. Tumor tissue sections were de-waxed in xylene twice for 10 minutes. Xylenes were removed through two 5 minute incubations in 100% ethanol. Tissues were re-hydrated by serial incubations in 95%, 85%, 70%, 50% and 30% ethanol made in saline. The tissues were re-fixed in 4% PFA/PBS for 20 minutes, and then washed twice in PBS. Next, tissues were treated with 20 μg/ml of proteinase K (Invitrogen) for 7.5 minutes, followed by a wash in PBS, another fixation in 4% PFA/PBS, and a final PBS wash. To prevent non-specific binding of probe, the tissues were twice acetylated for 5 minutes in 0.1 M triethanolamine-HCl containing 500 ul of acetic anhydride and 448 μl of 10N NaOH. After 5 minute incubations in PBS followed by saline, tissues were dehydrated through an inverse rehydration process (described above) and air-dried. Radio-labeled probe was placed in hybridization mixture (50% deionized formamide, 0.3 M NaCl, 20 mM Tris-HCl pH 8.0, 5 mM EDTA, 10 mM NaPO₄ pH 8.0, 10% dextran sulfate, 1×Denhardt's solution, 0.5 mg/ml yeast tRNA and 10 mm DTT) to a final concentration of 1.5×10⁵ cpm/μl and denatured at 80° C. for 2 min. Prepared tissue sections were covered with 60 μl of probe/hybridization mixture under a cover slip and allowed to incubate in a sealed container ON at 55° C. Cover slips were removed with a brief incubation in 5×SSC/0.1% 2-mercaptoethanol (2-ME) at 55° C. Slides were then placed in 50% formamide/2×SSC/2-ME at 65° C. for 30 minutes. Next, slides were washed 3 times in 0.5 M NaCl/10 mM Tris-HCl/5 mM EDTA at 37° C. followed by incubation in 20 μg/ml RNAse A (Roche) for 30 minutes in the same buffer. After a final wash in NaCl/Tris-HCl/EDTA buffer, a repeat high-stringency incubation in formamide/2×SSC/2-ME was performed for 30 minutes. Slides were then washed in 2×SSC/2-ME followed by 0.1×SSC/2-ME for 30 minutes each at 65° C. Finally, the tissue sections were dehydrated as described above. Slides were treated with Kodak NTB-2 nuclear emulsion and stored at 4° C. for approximately three weeks prior to development. Slides were developed in Kodak D-19 solution, fixed in Kodafix and counter-stained with 0.1% toluidine blue.

Quantification of Gene Expression. The quantification of radioactive in-situ hybridization assays was performed. by determining the number of activated silver grains over a predetermined area for tissue slides that were dipped in photographic emulsion and exposed. A linear relationship exists between the level of radioactivity hybridized to a tissue specimen and the grain number determined either by an image analyzer or by manual counting (45). A manual counting technique was employed as follows: a microscope set-up with an optical grid pattern and a 40× oil emersion objective used to count grains over a given area (i.e. 4 small squares) in several representative areas of the tumor specimen. For each experiment performed, hybridization of a given probe to normal or non-tumor structures (such as ducts, lobules or blood vessels) was also assessed and used as a standard to accommodate for inter-experiment variation. This was possible for Notch1, 2, 3, 4, DII1, Jagged1 and Jagged2, which were expressed in non-tumor structures. For these probes, inter-experimental variation was accounted for, and tumors divided into low, medium or high expressers depending on where they fell within the range from lowest to highest expression levels. For all other probes, tumors were divided into low, medium or high expressers depending on where they fell within the range from lowest to highest expression levels, without reference to a standard. Expression levels between different mRNA targets could not be compared with this method because of inherent differences in binding kinetics for each probe.

For NIH tissue microarrays, darkfield 20× magnification views of tumor tissue sections were digitally photographed (8-bit gray scale). Exposure time was standardized for all photographs. Using Image-Pro Plus (Media Cybernetics Inc) image analysis software, the concentration of activated silver grains was determined at four locations over the tumor and an average obtained. For each probe, high expression (Hi) was defined as expression that fell within the highest quartile of the expression range.

Immunohistochemistry. For assessment of microvascular density, representative 4 micron paraffin sections of tumors 22 and 23 were stained for CD31 expression using the Ventana “Benchmark™” immunostaining system, utilizing the Protease 1 digestion protocol and incubation with primary. anti-CD31 antibody (DAKO) for 32 minutes. The sections, counterstained with hematoxylin, were assessed morphologically and vascular “hot-spots” showing the greatest density of CD31 positive structures were selected. Within these areas, 10 fields at 40× were photographed. Using Image-Pro® Plus image analysis software (Media Cybernetics), the perimeter of vessels defined by CD31 staining was traced, and the total microvascular area (in square microns), was calculated for each photograph.

Statistical analysis. Predicted 10-year risks of mortality and relapse were calculated using Adjuvant! for each UHN breast cancer specimen for which all requested pathological parameters were available. Means, standard deviations and medians were calculated for the specimens with high expression of JAG1, NOTCH1, or NOTCH3 and specimens with low expression. These groups were compared using Mann-Whitney tests. For the 192 invasive primary ductal breast cancer samples obtained from the NCl, overall survival was measured from diagnosis to death or last follow-up. Kaplan-Meier curves were calculated for the high and low expression JAG1, NOTCH1, and NOTCH3 groups. Survival between groups was compared using the log-rank test. The co-expression of high-levels of JAG1 and NOTCH1 was similarly investigated. Cox proportional hazard regression was used to look for a dose-response relationship between level of gene expression and survival. Bi-variate models examined whether gene expression had an independent effect on survival after controlling for known predictors. Co-expression of high levels of JAG1 and NOTCH1, JAG1 and NOTCH3, and NOTCH1 and NOTCH3 were examined in contingency tables and tested for independence using the Chi-square test. P-values ≦0.05 were considered statistically significant.

### Example 1

### Patients and Tumor Samples

Tumor tissue examined in this study was obtained from patients undergoing surgery for palpable tumors greater than 2 cm in diameter at the University Health Network (Toronto, Ontano) between December 2002 and June 2004. Overall patient and tumor characteristics are shown in Tables 1 and 2. Tumors were examined for mRNAs encoding Notch1, 2, 3 and 4, Jagged 1 and 2, Delta 1, 3 and 4, Pref 1/DIK and Manic, Radical and Lunatic Fringe. Probes used are shown in Table 3. For each gene, from 20 to 50 invasive ductal carcinomas (IDC) were surveyed, as well as several invasive lobular carcinomas (ILC) and carcinomas in situ (CIS) (Table 2). For most genes, mRNA expression levels were analyzed in 20 tumors. Due to limitations of specimen size, not all probes could be used on the same samples. For Jagged1, Notch1, Notch3 and Pref1/DIk, up to 50 tumors were screened.

### Generation of Notch Activation System Probes and Optimization of RNA In Situ Hybridization for Human Breast Cancer Specimens

In order to examine expression of the Notch activation system in human breast cancer, expression vectors were generated to allow synthesis of ³³P-labeled antisense probes for all four Notch receptors (Notch1, 2, 3, 4), five Notch ligands (Delta-like or DII1, 3, 4, Jagged1, 2), one potential ligand (Pref1/DIk), and three Fringes (Lunatic Fringe, Manic Fringe and Radical Fringe). Breast tumors were procured and placed into RNAse-free fixative within 15 minutes of surgical resection. To ensure that RNA was intact and detectable via in situ hybridization, techniques were first optimized using an antisense E-cadherin probe. 16 of 18 IDC and 0 of 2 ILC expressed E-cadherin mRNA (Table 4). Furthermore, when E-cadherin sense probe was used as a negative control, there was no detectable signal.

### Analysis of Notch Receptor Gene Expression in Breast Cancer

Breast tumors were tested for expression of mRNAs encoding all four Notch receptors. Low-level Notch1 mRNA expression was observed in the normal duct and lobule epithelium found in some samples. Similar low-level Notch1 expression was seen in about half of the CIS (2/4), IDC (17/37) and ILC (2/5) samples, with high-level expression seen in 1 of 4 CIS and in 5 of 37 IDC (Table 4). Very low-level Notch1 expression was also seen in the endothelium of some blood vessels.

Notch2 expression was seen in 100% of CIS, IDC and ILC (Table 4) examined. In 2 of 4 CIS, and 18/20 IDC, Notch2 mRNA was expressed at high levels (Table 4). Notch2 was also highly expressed in 1 of 2 ILC. When normal tissues were examined, low-level Notch2 mRNA expression was observed in both ducts and lobules but the specific cell type of expression was not established. These data suggest that Notch2 signaling may occur in the normal breast and in breast cancers.

In normal breast tissue, Notch3 expression was observed in luminal epithelial cells. Similar low-level Notch3 mRNA expression was seen in 1 of 8 CIS samples, in 14 of 47 IDC, and in 1 of 6 ILC (Table 4). High-level Notch3 expression was seen in 3 of 8 CIS and in 6 of 47 IDC (Table 4). In 3 of 6 samples where CIS and invasive carcinoma coexisted, Notch3 expression was significantly reduced in the invasive component, as compared with the CIS component. In the other 3 cases, Notch3 was not detected in either invasive or in situ components of the tumor. In addition, Notch3 mRNA expression was present in the vascular smooth muscle cell (VSMC) layer of blood vessels and was absent from endothelial cells.

When Notch4 was studied, low-level expression was observed in 1 of 4 CIS, in 10 of 20 IDC and in no ILC studied (Table 4). High-level Notch4 expression was detected in only 1 of 20 IDC (Table 4). Recently, Imatani and Callahan (46) identified a novel 1.8 kb Notch4/Int3 mRNA species (designated h-Int3sh) that is expressed in a number of transformed human breast tumor cell lines. The design of our Notch4 antisense RNA probe was such that it should detect h-Int3sh, as well as full length Notch-4 mRNA (Table 3). Notch 4 expression was seen in breast cancer-associated vessels.

### Analysis of Notch Ligand Gene Expression in Breast Cancer

The Notch ligand DLL1 was expressed in normal lobules and ducts. Low-level DLL1 expression was noted in 2 of 4 CIS samples, in 6 of 20 IDC, and in 2 of 2 ILC (Table 4). High-level DLL1 expression was also seen in 2 of 20 IDC (Table 4). DLL3 and DLL4 signal was detected at low levels in a fraction of tumors (Table 4). No tumors expressed high levels of these ligands. DLL4 expression was observed in blood vessel endothelium.

Jagged1 expression was detected in normal myoepithelium. In breast, tumors, Jagged1 was expressed at low levels in 8 of 44 IDC, and in 1 of 5 ILC (Table 4). It was highly expressed in 1 of 7 CIS, and in 6 of 44 IDC (Table 4). Interestingly, three of these cases displayed a pattern of expression in which individual cells were highly variable in their levels of Jagged1 mRNA (data not shown). In these tumors, approximately 15% of tumor cells expressed extremely high levels of Jagged1 mRNA. Interestingly, 4 of the 6 tumors that express high levels of Jagged1 also express high levels of Notch3 mRNA in most tumor cells (Table 2). Jagged1 was also expressed in blood vessel endothelium. Low-level Jagged2 expression was observed in normal ducts and lobules, in 1 of 4 CIS, in 6 of 20 IDC (Table 4), and in blood vessel endothelium). High-level Jagged2 expression was seen in 1 of 4 CIS, and in 9 of 20 IDC (Table 4).

A gene known as Pref1 or DIk produces a protein with similarity to DII ligands (47). This gene has been implicated in regulation of adipogenesis in vivo (48). Low-level expression of this gene was seen in 3 of 40 IDC (Table 4). High expression was seen in 2 of 2 CIS, and in 4 of 40 IDC (Table 4). The pattern of Pref1/DIk expression was unusual in both samples of CIS. It was only seen in a subset of ductal structures filled with intraductal carcinoma, and in adjacent ducts only on the side nearest to the Pref1/DIk expressing duct. This suggests that this gene may be turned on in response to the secretion of a stromally produced hormone or growth factor. Interestingly, Pref1/DIk positive tumors were all grade III, PR negative, and three of seven were HER-2/neu positive.

### Analysis of Fringe Gene Expression in Breast Cancer

As Fringe proteins regulate Notch activation through control of ligand sensitivity, we examined expression of Fringe genes in human breast cancer (Table 4). Low expression of Lunatic Fringe was seen in 1 of 4 CIS, in 8 of 20 IDC and 0 of 2 ILC. High-level Lunatic Fringe was noted in 4 of 20 IDC. Low-level Manic Fringe was seen in 6 of 20 IDC. High-level expression of this Fringe was not observed in any of the tumor samples assessed. Low-level Radical Fringe expression was seen in normal ducts and lobules, in 3 of 4 expression was seen in 1 of 4 CIS and 5 of 20 IDC.

### Notch Signaling Component Expression in Tumor Blood Vessels

The Notch activation system is highly expressed in tumor-associated blood vessels (Table 4). The specific Notch receptors and ligands expressed in tumor-associated vessels were Notch1, 3, and 4, as well as DII4, Jagged 1, and Jagged2. Notch3 expression was confined to the vascular smooth muscle cell (VSMC) layer of breast tumor neovessels, including small arterioles. Together, Notch3 antisense probe, Jagged1 antisense probe, anti-smooth muscle antibody, and anti-CD31 (CD31/Pecam-1 is an endothelial cell surface marker) all clearly demonstrated a rich network of blood vessels between ductal lesions. In ductal CIS (DCIS), two distinct vascular patterns are observed: a diffuse increase in stromal vascularity between duct lesions (so-called pattern I), and a dense rim of microvessels adjacent to the basement membrane of individual ducts (pattern II). Tumors can exhibit either a single pattern, or both patterns together. One DCIS sample examined demonstrated pattern II vascular distribution using a Notch3 probe to identify the VSMC layer of blood vessels. Similar pattern II distribution was shown using a Jagged1 antisense probe to mark blood vessel endothelium.

Invasive ductal carcinomas varied in their distribution of Notch3-positive neovessels. Tumor 22, for example, contained relatively few Notch3-positive blood vessels, whereas Tumor 23 was richly populated with Notch-3 positive blood vessels. This difference could be due to elevated VSMC Notch3 expression and/or due to a greater blood vessel volume in tumor 23. To explore these possibilities, areas of maximal vascular density in tumors 22 and 23 were assessed for activated silver grain density to quantitate Notch3 mRNA expression levels. Tumor 23 exhibited approximately 8 times greater Notch3 expression compared with tumor 22. Tumor microvascular density was assessed using CD31 staining and analysis of total vascular area using Image-Pro® Plus software (see Materials and Methods). The microvascular density of tumor 23 was found to be approximately 10 times greater than tumor 22 (comparable to 8 times greater Notch3 expression), suggesting that the elevated Notch3 mRNA signal was due to increased vascular density alone.

### Example 2

### Correlation of Notch Receptor (Notch1 and 3) and Ligand (Jagged1) Expression with Poor Pathological Prognostic Features in Breast Cancer

To directly test for expression of Notch ligands (JAG1, JAG2, DLL1, DLL3 and DLL4) and receptors (NOTCH1, 2, 3, and 4) in human breast cancer, as well as in associated blood vessels and stroma, we used mRNA in situ hybridization. Our initial screen was performed on tumors greater than 2 cm in diameter obtained from patients at the University Health Network (UHN—Toronto, Ontario) (Table 1). Within this cohort we identified a group of tumors that co-expressed very high levels of Notch ligand, Notch receptor and, in some cases, Fringe gene mRNA (Table 4). Therefore, we analyzed Notch ligand and receptor gene expression in a group of up to 50 tumors, and tested for correlations between expression and pathological data. The DLL1, JAG1, and JAG2 ligands were expressed at very high levels in 2/22, 6/47 and 9/22 tumors respectively (Table 4). The Pref1/DLK gene, which may encode a DLL-family Notch ligand, was expressed at high levels in 4 of 42 tumors. Notch receptor genes were also expressed at high levels in a variable number of breast tumors: 5/39 tumors expressed high levels of NOTCH1, 19/22 tumors expressed high levels of NOTCH2, 6/50 tumors expressed high levels of NOTCH3, and 1/22 tumors expressed high levels of NOTCH4 (Table 4). Finally, LUNATIC FRINGE, MANIC FRINGE and RADICAL FRINGE were expressed at high levels in 4/20, 0/20, and 5/20 tumors respectively. A number of ligands and receptors were expressed at high levels in tumor-associated vasculature (Table 4). In addition, some of the tumor samples contained areas of normal mammary tissue, and in these cases we saw NOTCH3 expression in luminal epithelial cells and JAG1 expression in the surrounding myoepithelial layer, suggesting that this ligand/receptor pair may normally interact in this context.

We next tested for an association between Notch ligand or receptor gene-expression and 10-year risk of mortality or relapse calculated using Adjuvant!, a widely used clinical tool to predict the risk of negative outcome based on tumor pathological features and patient characteristics (www.adjuvantonline.com) (Ravdin et al., Journal of Clinical Oncology, 19:980-991 (2001)). When tumors were grouped into low expressers and high expressers for each gene, and pathological data analyzed for predicted mortality and relapse in each group, we observed a statistically significant relationship between high-level JAG1 expression and increased predicted mortality when compared with tumors expressing low levels (median predicted mortality 63% for JAG1^{Hi} vs. 32% for JAG1^{Lo} p=0.04) (Table 5). Similarly, we observed statistically significant relationships between high-level NOTCH1 or NOTCH3 expression and increased predicted mortality when compared with tumors expressing low levels of these receptors (median predicted mortality 66% for NOTCH1^{Hi} vs. 30.5% for NOTCH1^{Lo} p=0.005 and median predicted mortality 55.5% for NOTCH3^{Hi} vs. 31.5% for NOTCH3^{Lo} p=0.02) (Table 5). Predicted relapse data showed the same trends, although conventional significance was only reached for NOTCH1 expression (median predicted relapse 71.5% for JAG1 Hi vs. 51% for JAG1^{Lo} p=0.06, predicted relapse 74% for NOTCH1^{Hi} vs. 52% for NOTCH1^{Lo} p=0.004, and predicted relapse 71% for NOTCH3Hi vs. 52% for NOTCH3^{Lo} p=0.09) (Table 3). Interestingly, the tumors with high levels of JAG1, NOTCH1 or NOTCH3 did not express high levels of erbB2 (Table 2).

### Example 3

The expression of JAG1, NOTCH1 and NOTCH3 was analysed in a large panel of breast cancers with associated patient follow up data. Tissue microarrays (TMA) were obtained from the US National Cancer Institute Cooperative Breast Cancer Tissue Resource (CBCTR). These TMAs were constructed from a cohort of tumors that were ⅓ node-negative, ⅓ node positive and ⅓ metastatic (n=64 for each group). We performed in situ hybridization to analyze tumor-specific expression. For these experiments, the system was modified for gene expression quantitation by using image analysis software-to determine the concentration of activated silver grains in multiple areas of tumor for each sample, as described above. Expression data for JAG1, NOTCH1 and NOTCH3 are tabulated together with data on individual patient and tumor characteristics (Table 6).

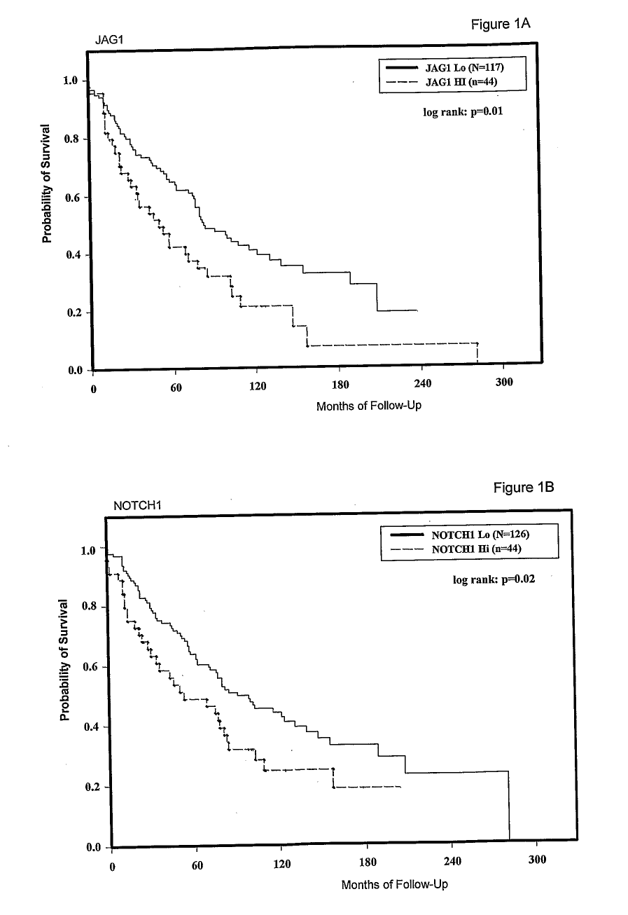

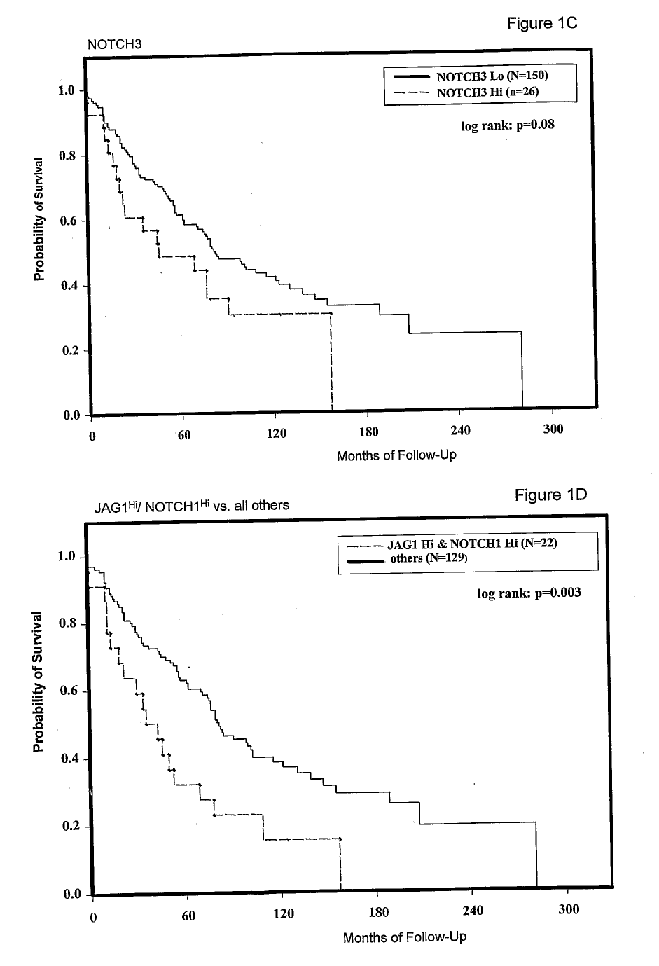

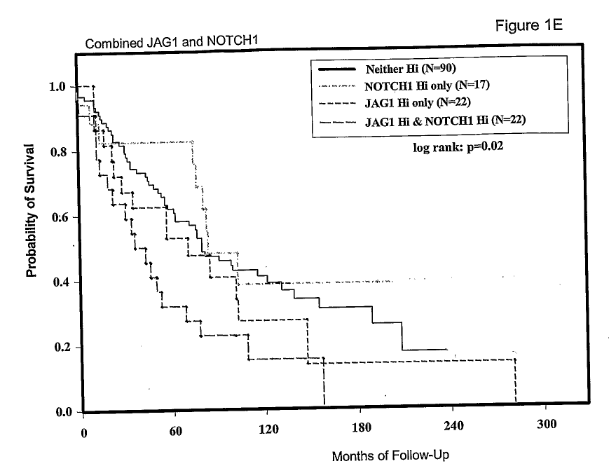

We first tested for relationships between expression of each gene and overall patient survival. JAG1 expression data showed a dose-dependent relationship with negative outcome. For example, patients with tumors expressing JAG1 at levels within the top 25% of the expression range were 37% more likely to die (Hazard ratio over interquartile range of 1.37, p=0.02) than patients with tumors expressing JAG1 at levels in the bottom quartile expression group (Table 7a). As expected, patients with JAG1^{Hi} tumors (those expressing JAG1 in the top quartile of the expression range) had reduced overall survival compared to JAG1^{Lo} tumors (expressing JAG1 at levels within the bottom three quartiles of the expression range) (5-year survival rates of 42% vs. 65%), with a median survival time of 50 months as compared to 83 months (p=0.01) (Table 7b and FIG. 1a). Furthermore, high JAG1 expression was found to be an independent predictor of poor outcome in bi-variate analyses with other known predictors of outcome including metastases, patient age, tumor size, node status, ER positivity, and tumor grade (Table 8). Similarly, patients with NOTCH1^{Hi} tumors had reduced overall survival compared to NOTCH1^{Lo} tumors (5-year survival rates of 49% vs. 64%), with a median survival time of 53 months as compared to 91 months (p 0.02) (Table 7b and FIG. 1b). A similar trend was observed for NOTCH3, although it did not reach statistical significance (p=0.08) (Table 7b and FIG. 1c).

The levels of expression of JAG1 and NOTCH1 and/or NOTCH3 receptors were not independent of each other. More tumors than expected by random chance co-expressed high levels of JAG1 and either receptor (p=0.001; Table 9). We therefore tested for any relationship between high-level co-expression of JAG1 and NOTCH receptors and patient survival. Indeed, patients harboring tumors with high-level JAG1 and high-level NOTCH1 (J1^{Hi} N1^{Hi}) had demonstrated worse overall survival than the patients with JAG1^{Hi} or —NOTCH1^{Hi} tumors described above, or indeed than all other patients (32% 5-year survival and 40 months median survival for J1^{Hi} N1^{Hi} vs. all other patients with 63% 5-year survival and 81 months median survival, p=0.003) (Table 7b and FIG. 1d). Furthermore, subgroup analysis suggested that in comparison to patients with tumors expressing low levels of both JAG1 and NOTCH1 (J1^{Lo} N1^{Lo}) or high levels of either JAG1 (J1Hi N1^{Lo}) or NOTCH1 (J1^{Lo} N1^{Hi}), tumors co-expressing high levels of JAG1 and NOTCH1 demonstrated worse overall survival (Table 7b and FIG. 1e). Interestingly, these data also suggest the existence of two types of NOTCH1^{Hi} tumors, those that co-express high levels of JAG1 and those with low levels of JAG1.

### Example 4

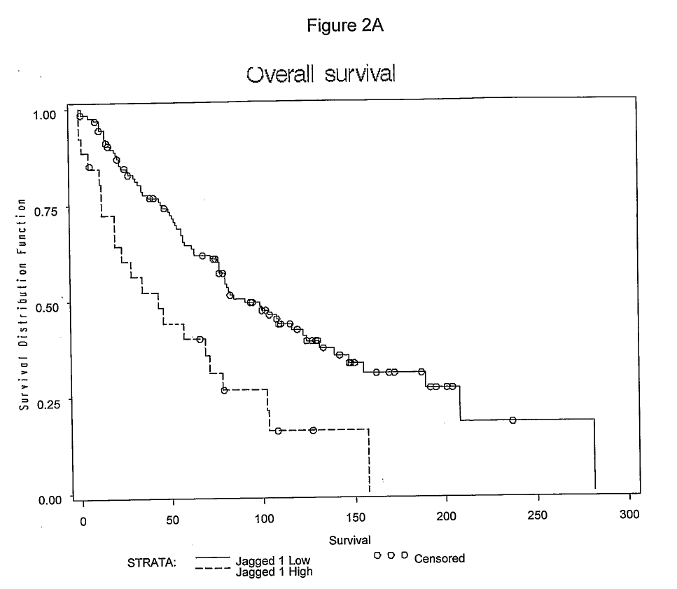

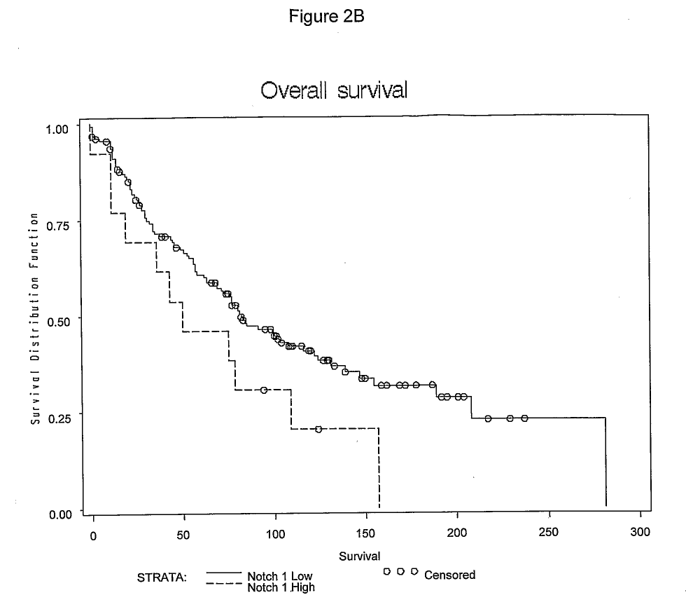

As an alternative to quantification by counting silver grains by image analysis to assess gene expression levels, a more rapid technique was adapted from the method described by Allred et al. (56). Using this method, the proportion of cells positive for silver grains is scored from 0 to 5 (5 being greater than ⅔ of cells positive) and is added to the average density of silver grains over positive cells scored from 0 to 3 (3 being highest in the density range). This method was applied to the samples described in Example 3 and the results are shown in Tables 9 and 10 and FIGS. 2A and 2B.

Patents with tumors expressing JAGGED 1 (FIG. 2A and Table 9) in the top quartile of the expression range had a significantly poorer 5 year survival and median survival than patients with tumor expression in the bottom 3 quartiles (p<0.001). Similarly, patients with tumors expressing NOTCH 1 (FIG. 2B and Table 10) in the top quartile of the expression range had a poorer 5 year and median survival than patients with tumor expression in the bottom 3 quartiles (p=0.08).

### REFERENCES

1. Society, A. C. 2000. ACS Cancer facts and figures. Cancer Practise 8:105-107.
2. Slamon, D. J., Godolphin, W., Jones, L. A., Holt, J. A., Wong, S. G., Keith, D. E., Levin, W. J., Stuart, S. G., Udove, J., Ullrich, A., et al. 1989. Studies of the HER-2/neu Proto-oncogene in Human Breast and Ovarian Cancer. Science 244:707-712.
3. Borg, A., Tandon, A. K., Sigurdsson, H., Clark, G. M., Ferno, M., Fuqua, S. A., Killander, D., and McGuire, W. L. 1990. HER-2/neu amplification predicts poor survival in node-positive breast cancer. Cancer Res 50:4332-4337.
4. Cleton-Jansen, A. M. 2002. E-cadherin and loss of heterozygosity at chromosome 16 in breast carcinogenesis: different genetic pathways in ductal and lobular breast cancer Breast Cancer Res 4:5-8.
5. van 't Veer, L. J., Dai, H., van de Vijver, M. J., He, Y. D., Hart, A. A., Bernards, R., and Friend, S. H. 2003. Expression profiling predicts outcome in breast cancer. Breast Cancer Res 5:57-58.
6. Callahan, R., and Smith, G. H. 2000. MMTV-induced mammary tumorigenesis: gene discovery, progression to malignancy and cellular pathways. Oncogene 19:992-1001.
7. Brennan, K. R., and Brown, A. M. 2004. Wnt proteins in mammary development and cancer. J Mammary Gland Biol Neoplasia 9:119-131.
8. Dickson, C., Spencer-Dene, B., Dillon, C., and Fantl, V. 2000. Tyrosine kinase signaling in breast cancer: fibroblast growth factors and their receptors. Breast Cancer Res 2:191-196.
9. Dillon, C., Spencer-Dene, B., and Dickson, C. 2004. A crucial role for fibroblast growth factor signaling in embryonic mammary gland development. J Mammary Gland Biol Neoplasia 9:207-215.
10. Robbins, J., Blondel, B. J., Gallahan, D., and Callahan, R. 1992. Mouse mammary tumor gene int-3: a member of the notch gene family transforms mammary epithelial cells. J Virol 66:2594-2599.
11. Egan, S. E., St-Pierre, B., and Leow, C. C. 1998. Notch receptors, partners and regulators: from conserved domains to powerful functions. Curr Top Microbiol Immunol 228:273-324.
12. Artavanis-Tsakonas, S., Rand, M. D., and Lake, R. J. 1999. Notch signaling: cell fate control and signal integration in development. Science 284:770-776.
13. Callahan, R., and Egan, S. E. 2004. Notch signaling in mammary development and oncogenesis. J Mammary Gland Biol Neoplasia 9:145-163.
14. Haltiwanger, R. S., and Stanley, P. 2002. Modulation of receptor signaling by glycosylation: fringe is an O-fucose-beta1,3-N-acetylglucosaminyltransferase. Biochim Biophys Acta 1573:328-335.
15. Haines, N., and Irvine, K. D. 2003. Glycosylation regulates Notch signaling. Nat Rev Mol Cell Biol 4:786-797.
16. Kopan, R. 2002. Notch: a membrane-bound transcription factor. J Cell Sci 115:1095-1097.
17. Lai, E. C. 2002. Keeping a good pathway down: transcriptional repression of Notch pathway target genes by CSL proteins. EMBO Rep 3:840-845.
18. Yamamoto, N., Yamamoto, S., Inagaki, F., Kawaichi, M., Fukamizu, A., Kishi, N., Matsuno, K., Nakamura, K., Weinmaster, G., Okano, H., et al. 2001. Role of Deltex-1 as a transcriptional regulator downstream of the Notch receptor. J Biol Chem 276:45031-45040.
19. Crowner, D., Le Gall, M., Gates, M. A., and Giniger, E. 2003. Notch steers Drosophila ISNb motor axons by regulating the AbI signaling pathway. Curr Biol 13:967-972.
20. Bellavia, D., Campese, A. F., Alesse, E., Vacca, A., Felli, M. P., Balestri, A., Stoppacciaro, A., Tiveron, C., Tatangelo,L., Giovarelli, M., et al. 2000. Constitutive activation of NF-kappaB and T-cell leukemia/lymphoma in Notch3 transgenic mice. Embo J 19:3337-3348.
21. Axelrod, J. D., Matsuno, K., Artavanis-Tsakonas, S., and Perrimon, N. 1996. Interaction between Wingless and Notch signaling pathways mediated by Dishevelled. Science 271:1826-1832.
22. Kamakura, S., Oishi, K., Yoshimatsu, T., Nakafuku, M., Masuyama, N., and Gotoh, Y. 2004. Hes binding to STAT3 mediates crosstalk between Notch and JAK-STAT signaling. Nat Cell Biol 6:547-554.
23. Blokzijl, A., Dahlqvist, C., Reissmann, E., Falk, A., Moliner, A., Lendahl, U., and Ibanez, C. F. 2003. Cross-talk between the Notch and TGF-beta signaling pathways mediated by interaction of the Notch intracellular domain with Smad3. J Cell Biol 163:723-728.
24. Sade, H., Krishna, S., and Sarin, A. 2004. The anti-apoptotic effect of Notch-1 requires p56Ick-dependent, Akt/PKB-mediated signaling in T cells. J Biol Chem 279:2937-2944.
25. Nair, P., Somasundaram, K., and Krishna, S. 2003. Activated Notch1 inhibits p53-induced apoptosis and sustains transformation by human papillomavirus type 16 E6 and E7 oncogenes through a PI3K-PKB/Akt-dependent pathway. J Virol 77:7106-7112.
26. Djonov, V., Andres, A. C., and Ziemiecki, A. 2001. Vascular remodelling during the normal and malignant life cycle of the mammary gland. Microsc Res Tech 52:182-189.
27. Haslam, S. Z., and Woodward, T. L. 2003. Host microenvironment in breast cancer development: epithelial-cell-stromal-cell interactions and steroid hormone action in normal and cancerous mammary gland. Breast Cancer Res 5:208-215.
28. Wiseman, B. S., and Werb, Z. 2002. Stromal effects on mammary gland development and breast cancer. Science 296:1046-1049.
29. Jhappan, C., Gallahan, D., Stahle, C., Chu, E., Smith, G. H., Merlino, G., and Callahan, R. 1992. Expression of an activated Notch-related int-3 transgene interferes with cell differentiation and induces neoplastic transformation in mammary and salivary glands. Genes Dev 6:345-355.
30. Smith, G. H., Gallahan, D., Diella, F., Jhappan, C., Merlino, G., and Callahan, R. 1995. Constitutive expression of a truncated INT3 gene in mouse mammary epithelium impairs differentiation and functional development. Cell Growth Differ 6:563-577.
31. Gallahan, D., Jhappan, C., Robinson, G., Hennighausen, L., Sharp, R., Kordon, E., Callahan, R., Merlino, G., and Smith, G. H. 1996. Expression of a truncated Int3 gene in developing secretory mammary epithelium specifically retards lobular differentiation resulting in tumorigenesis. Cancer Res 56:1775-1785.
32. Callahan, R., and Raafat, A. 2001. Notch signaling in mammary gland tumorigenesis. J Mammary Gland Biol. Neoplasia 6:23-,36.
33. Uyttendaele, H., Soriano, J. V., Montesano, R., and Kitajewski, J. 1998. Notch4 and Wnt-1 proteins function to regulate branching morphogenesis of mammary epithelial cells in an opposing fashion. Developmental Biology 196:204-217.
34. Dievart, A., Beaulieu, N., and Jolicoeur, P. 1999. Involvement of Notch1 in the development of mouse mammary tumors. Oncogene 18:5973-5981.
35. Kiaris, H., Politi, K., Grimm, L. M., Szabolcs, M., Fisher, P., Efstratiadis, A., and Artavanis-Tsakonas, S. 2004. Modulation of notch signaling elicits signature tumors and inhibits hras1-induced oncogenesis in the mouse mammary epithelium. Am J Pathol 165:695-705.
36. Parr et al., (2004), Int. J. Molec. Medicine, v.14, pp. 779-786.
37. Ellissen et al., (1991), Cell 66:649-661.
38. Joutel et al., (1996), Nature 383:707-710.
39. Li et al., (1997), Nature Genetics 16:243-251.
40. Mumm et al., (2000), Molec. Cell, 5, 197-206.
41. Anderson et al., (2005), Biochem. Pharmacol., 69, 689-698.
42. Tomita et al., (2004), Drug News Perspect., 17, 321-325.
43. Saxena et al., (2001), J. Biol. Chem., 276, 40268-73.
44. Pece et al., (2004), J. Cell Biol., 167, 215-221.
45. Swan, M. C., Najlerahim, A. R., and Bennett, J. P. 1997. Expression of serotonin transporter mRNA in rat brain: presence in neuronal and non-neuronal cells and effect of paroxetine. J Chem Neuroanat 13:71-76.
46. Imatani, A., and Callahan, R. 2000. Identification of a novel NOTCH-4/INT-3 RNA species encoding an activated gene product in certain human tumor cell lines. Oncogene 19:223-231.
47. Villena, J. A., Kim, K. H., and Sul, H. S. 2002. Pref-1 and ADSF/resistin: two secreted factors inhibiting adipose tissue development. Horm Metab Res 34:664-670.
48. Moon, Y. S., Smas, C. M., Lee, K., Villena, J. A., Kim, K. H., Yun, E. J., and Sul, H. S. 2002. Mice lacking paternally expressed Pref-1/DIk1 display growth retardation and accelerated adiposity. Mol Cell Biol 22:5585-5592.
49. Lanz, T. A., Hosley, J. D., Adams, W. J., and Merchant, K. M. 2004. Studies of Abeta pharmacodynamics in the brain, cerebrospinal fluid, and plasma in young (plaque-free) Tg2576 mice using the gamma-secretase inhibitor N2-[(2S)-2-(3,5-difluorophenyl)-2-hydroxyethanoyl]-N1-[(7S)-5-methyl-6-oxo-6,7-dihydro-5H-dibenzo[b,d]azepin-7-yl]-L-alaninamide (LY-411575). J Pharmacol Exp Ther 309:49-55.
50. Perou et al., (2000), Nature, 406, 747-752
51. Sorlie et al., (2001), PNAS (USA), 98, 10869-10874
52. Van't Veer et al., (2002), Nature, 416, 530-536
53. Kipshidze et al., (2005), Expert Opin. Biol. Ther., 5, 79-89.
54. Hotz et al., (2005), Surgery, 137, 192-9.

55. Takabatake et al., (2005), Gene Ther., February 24, E-publication.
56. Allred et al., (1998), Mod. Pathol., v. 11, pp. 155-168
57. Stylianou et al., (2006), Cancer Res., v. 66, pp. 1517-1525
58. Burbulis et al., (2005), Nat. Methods, v. 2, pp. 31-37
59. Ausubul et al., eds., (1995) “Current Protocols in Molecular Biology”, Wiley & Sons, N.Y.

TABLE 1
Patient Characteristics

| | UHN | NCI |
|---|---|---|
| N | 51 | 184 |
| Age¹ | 59 (29-86) | 59 (29-89) |
| Tumour size (cm)¹ | 3.7 (1.2-18) | 2.3 (0.1-6) |
| Grade (%) | | |
| I | 12 | 17 |
| II | 25 | 64 |
| III | 63 | 18 |
| Node positive (%) | 57 | 59 |
| Necrosis (%) | 34 | — |
| LVI (%) | 49 | — |
| ER positive (%) | 67 | 65 |
| PR positive (%) | 45 | 33 |
| c-ERB2/neu positive (%) | 19 | — |

¹Mean (Range)

TABLE 2

| | Patient | | | | | | | | | | | |
|---|---|---|---|---|---|---|---|---|---|---|---|---|
| | 1 | 2 | 3 | 4 | 6 | 7 | 8 | 9 | 10 | 11 | 12 | 13 |
| Age | 80 | 48 | 38 | 56 | 51 | 66 | 75 | 69 | 51 | 65 | 83 | 76 |
| Tumor(s) | lobular | lobular/duc | NOS | Metaplastic | NOS | NOS | NOS | NOS | NOS | NOS | NST | NST |
| Nodes | 0/9 | 2/19 | 0/22 | 0/15 | 0/5 | 5/14 | 0/18 | 0/4 | 3/19 | | 0/1 | 9/19 |
| Size(cm) | 3.2 | 1.5 | 2.4 | 3.2 | 2.2 | 1.5 | 2.5 | 2 | 8 | 1.9 | 1.7 | 3 |
| Grade | II | III | III | III | III | III | II | I | I | I | I | III |
| ER | + | + | + | − | + | − | + | | + | + | + | − |
| PR | − | + | + | − | − | − | + | | − | + | + | − |
| LVI | − | + | − | − | + | − | − | − | + | − | − | + |
| Necrosis | − | − | − | + | − | − | − | − | − | − | − | + |
| Her-2/neu | − | − | − | − | − | − | − | | − | − | − | − |
| DLL1 | 1 | 1 | 1 | | 2 | 2 | | 0 | 0 | 0 | 0 | 0 |
| DLL3 | 0 | 0 | | | 0 | 0 | 0 | 0 | 0 | 0 | 0 | 0 |
| DLL4 | 1 | 0 | 1 | | 1 | 1 | 0 | 1 | 1 | 1 | 0 | 1 |
| JAG1 | 1 | | 0 | | 0 | 2 | 0 | 0 | 2 | 1 | 0 | 2 |
| JAG2 | 0 | 0 | 0 | | 0 | 2 | 1 | 1 | | 1 | 0 | 1 |
| NOTCH 1 | 0 | 0 | 1 | | 1 | 2 | 0 | 0 | | 0 | 0 | 1 |
| NOTCH 2 | 2 | 1 | 2 | | 2 | 2 | 2 | 2 | 2 | 2 | 1 | 2 |
| NOTCH 3 | 0 | 0 | 0 | 1 | 0 | 1 | 0 | 1 | 1 | 0 | 0 | 2 |
| NOTCH 4 | 0 | 0 | 0 | | 1 | 1 | 1 | 0 | | 1 | 1 | 1 |
| CK 5 | | | | | | + | | | + | | | ‘+/− |

| | Patient | | | | | | | | | | | | |
|---|---|---|---|---|---|---|---|---|---|---|---|---|---|
| | 14 | 15 | 16 | 17 | 18 | 19 | 20 | 21 | 22 | 23 | 24 | 25 | 26 |
| Age | 53 | 63 | 72 | 62 | 41 | 84 | 59 | 57 | 62 | 71 | 86 | 50 | 72 |
| Tumor(s) | NOS | NOS | NOS | NOS | NOS | NOS | metaplastic | micropap | NOS | NOS | NOS | NOS | NOS |
| Nodes | 0/4 | 0/13 | 2/10 | 10/26 | 5/14 | ND | 4/22 | 2/14 | 0/28 | 1/11 | ND | 2/17 | 10/16 |
| Size(cm) | 3.8 | 2 | 2.7 | 3.2 | 2.4 | 2.7 | 3.5 | 1.2 | 3.5 | 5 | 2.8 | 3.5 | 3.8 |
| Grade | III | II | I | III | III | II | III | III | II | II | III | II | II |
| ER | + | − | + | − | − | + | − | + | + | + | − | + | + |
| PR | − | − | + | − | − | + | − | − | + | + | − | + | + |
| LVI | + | − | − | − | + | − | + | + | + | + | + | − | + |
| Necrosis | − | − | + | − | + | − | + | − | + | + | + | ND | − |
| Her-2/neu | + | + | − | − | − | − | − | − | − | − | − | − | − |
| DLL1 | 1 | 1 | 0 | 0 | 1 | 1 | 0 | 0 | 0 | 0 | 0 | 0 | |
| DLL3 | 1 | 0 | 0 | 1 | 1 | 1 | 0 | 0 | 0 | | 0 | 0 | 0 |
| DLL4 | 1 | 1 | 0 | 1 | 1 | 1 | 0 | 0 | 0 | 0 | 0 | | |
| JAG1 | 1 | 0 | 1 | 0 | 0 | 1 | 2 | 1 | 1 | 1 | 2 | 0 | 0 |
| JAG2 | 2 | 2 | 1 | 2 | 2 | 2 | 2 | 2 | 2 | 1 | 0 | 0 | |
| NOTCH 1 | 1 | 0 | 0 | 1 | 2 | | 1 | 1 | 1 | 1 | 2 | 1 | 2 |
| NOTCH 2 | 2 | 2 | 2 | 2 | 2 | 2 | 2 | 1 | 2 | 2 | 2 | | |
| NOTCH 3 | 1 | 1 | 1 | 1 | 0 | 0 | 2 | 0 | 0 | 0 | 2 | 0 | 1 |
| NOTCH 4 | 1 | 1 | 0 | 1 | 2 | 1 | 0 | 0 | 0 | 0 | 0 | 0 | |
| CK 5 | | | | ‘+/− | + | | + | | | | + | − | − |

| | Patient | | | | | | | | | | | | |
|---|---|---|---|---|---|---|---|---|---|---|---|---|---|
| | 28 | 29 | 30 | 31 | 32 | 33 | 34 | 35 | 37 | 38 | 39 | 41 | 42 |
| Age | 72 | 73 | 46 | 55 | 49 | 62 | 52 | 43 | 64 | 65 | 62 | 45 | 48 |
| Tumor(s) | lobular | lobular | IDC | NOS | NOS | NOS | IDC/ILC | NOS | IDC/ILC | NOS | IDC | IDC | IDC |
| Nodes | 1/12 | 0/11 | 2/17 | 4/25 | 0/14 | 2/18 | 39/40 | 1/21 | 8/36 | 1/10 | ND | 1/12 | 0/14 |
| Size(cm) | 12 | 14 | 2.5 | 4.5 | 3 | 2.4 | 7.5 | 3 | 3 | 3 | 1.3 | 1.9 | 2.7 |
| Grade | I | II | III | III | III | II | III | III | II | III | III | III | III |
| ER | + | + | − | − | + | + | + | + | + | + | | + | − |
| PR | − | + | − | − | − | + | − | − | + | + | | − | − |
| LVI | − | + | + | − | + | − | ++ | − | ++ | − | − | − | + |
| | | | | | | | | | | | | | rare |
| Necrosis | − | − | + | + | + | − | − | + | − | − | − | − | − |
| Her-2/neu | − | − | − | + | − | + | − | − | + | − | | + | − |
| DLL1 | | | | | | | | | | | | | |
| DLL3 | | | | | | | | | | | | | |
| DLL4 | | | | | | | | | | | | | |
| JAG1 | 0 | 0 | 0 | 0 | 0 | 0 | 0 | 2 | 0 | 0 | 0 | 0 | 0 |
| JAG2 | | | | | | | | | | | | | |
| NOTCH 1 | 1 | | 0 | 0 | 0 | 1 | 0 | 1 | 1 | 0 | 0 | 1 | 0 |
| NOTCH 2 | | | | | | | | | | | | | |
| NOTCH 3 | 1 | 0 | 1 | 1 | 0 | 1 | 0 | 2 | 0 | 0 | 0 | 0 | 0 |
| NOTCH 4 | | | | | | | | | | | | | |
| CK 5 | | | | − | | | − | + | | − | | | |

| | Patient | | | | | | | | | | | | |
|---|---|---|---|---|---|---|---|---|---|---|---|---|---|
| | 43 | 45 | 46 | 47 | 48 | 49 | 50 | 51 | 52 | 53 | 54 | 55 | 56 |
| Age | 58 | 65 | 43 | 29 | 50 | 55 | 31 | 65 | 44 | 61 | 43 | 58 | 60 |
| Tumor(s) | IDC | NOS | IDC | IDC | IDC | IDC | NOS | NOS | NOS | NOS | IDC | NOS | NOS |
| Nodes | 0/20 | 0/9 | 20/24 | 0/5 | 0/14 | 0/21 | 0/18 | 4/16 | 4/13 | 0/1 | 2/27 | 3/25 | 3/12 |
| Size(cm) | 4 | 1.8 | 18 | 4.2 | 2.5 | 2.6 | 3.5 | 2.7 | 1.5 | 3.5 | 2.9 | 4 | 3.3 |
| Grade | III | III | III | III | III | II | III | III | III | III | III | III | II |
| ER | + | + | − | − | − | + | − | + | + | − | + | + | + |
| PR | +/− | + | − | − | − | + | − | + | + | − | + | − | + |
| LVI | − | − | + | + | − | − | − | + | + | − | + | + | + |
| Necrosis | − | − | + | + | − | + | + | − | − | + | − | − | − |
| Her-2/neu | − | − | − | − | − | − | FISH −/+ | + | − | | − | + | − |
| DLL1 | | | | | | | | | | | | | |
| DLL3 | | | | | | | | | | | | | |
| DLL4 | | | | | | | | | | | | | |
| JAG1 | 0 | 0 | 0 | 1 | 0 | 0 | 0 | 0 | 0 | 0 | 0 | | |
| JAG2 | | | | | | | | | | | | | |
| NOTCH 1 | 0 | 1 | 2 | | 1 | 1 | | | | | | | |
| NOTCH 2 | | | | | | | | | | | | | |
| NOTCH 3 | 0 | 0 | 0 | 2 | 0 | 0 | 1 | 1 | 0 | | 0 | 2 | 0 |
| NOTCH 4 | | | | | | | | | | | | | |
| CK 5 | | | | | | | | | | | | | |

TABLE 3

| Target mRNA | Length (bp) | Coding sequence (nts) | Probe (nts) |
|---|---|---|---|
| NOTCH1 | 7693 | 1-7671 | 5231-4606 |
| NOTCH2 | 11433 | 257-7672 | 443-92 |
| NOTCH3 | 8091 | 79-7044 | 6163-5489 |
| NOTCH4 | 6836 | 140-6148 | 6740-6210 |
| JAG1 | 5942 | 460-4116 | 1348-939 |
| JAG2 | 5077 | 405-4121 | 2456-1744 |
| DLL1 | 3162 | 323-2494 | 1756-1549 |
| DLL3 | 2347 | 17-1873 | 1288-642 |
| DLL4 | 3339 | 277-2334 | 3112-2673 |

Abbreviations:
bp, base pairs;
nts, nucleotides

TABLE 4
Expression of Notch signaling component mRNA in human breast cancers

| mRNA | | | | |
|---|---|---|---|---|
| probe | CIS | IDC | ILC | Vessels |
| E-cadherin | 1^{Lo} + 2^{Hi}/3 | 16^{Hi}/180/2 | | |
| NOTCH1 | 2^{Lo} + 1^{Hi}/4 | 17^{Lo} + 5^{HI}/37 | 2^{Lo}/5 | endothelium |
| NOTCH2 | 2^{Lo} + 2^{Hi}/4 | 2^{Lo} + 18^{Hi}/20 | 1^{Lo} + 1Hi/2 | |
| NOTCH3 | 1^{Lo} + 3^{Hi}/8 | 14^{Lo} + 6^{Hi}/47 | 1^{Lo}/6 | VSMC |
| NOTCH4 | 1^{Lo}/4 | 10^{Lo} + 1^{Hi}/20 | 0/2 | endothelium |
| DLL1 | 2^{Lo}/4 | 6^{Lo} + 2^{Hi}/20 | 2^{Lo}/2 | |
| DLL3 | 0/4 | 4^{Lo}/20 | 0/2 | |
| DLL4 | 2^{Lo}/4 | 12^{Lo}/201^{Lo}/2 | | endothelium |
| JAG1 | 1^{Hi}/7 | 8^{Lo} + 6^{Hi}/44 | 1^{Lo}/5 | endothelium |
| JAG2 | 1^{Lo} + 1^{Hi}/4 | 6^{Lo} + 9^{Hi}/20 | 0/2 | endothelium |
| Pref1/Dlk | 2^{Hi}/2 | 3^{Lo} + 4^{Hi}/40 | 0/4 | |
| Lunatic | 1^{Lo}/4 | 8^{Lo} + 4^{Hi}/20 | 0/2 | |
| Manic | 0/4 | 6^{Lo}/20 | 0/2 | |
| Radical | 3^{Lo} + 1^{Hi}/4 | 15^{Lo} + 5^{Hi}/20 | 2^{Lo}/2 | |

For Notch1, three tumors contained both ductal and lobular carcinoma and are therefore listed twice (once under ductal and once under lobular - i.e. the total number of tumors analyzed for Notch 1 is 39, and not 37 + 5).
For Notch3, two tumors contained both ductal and lobular carcinoma and are therefore listed twice (once under ductal and once under lobular - i.e. the total number of tumors analyzed for Notch 3 is 51, and not 47 + 6).
For Jagged1, two tumors contained both ductal and lobular carcinoma and are therefore listed twice (once under ductal and once under lobular). 47 total
For Pref-1, two tumors contained both ductal and lobular carcinoma and are therefore listed twice (once under ductal and once under lobular). 42 total
Numerators, 0 = no detectable expression above background, Lo = low expression levels, Hi = high expression levels
In cases where expression was detected in normal mammary cells, the Lo expression level was standardized to this normal level.

TABLE 5
Adjuvant! analysis of breast cancer patients from UHN

| | | N | Mean (STD) | Median | P-value¹ |
|---|---|---|---|---|---|
| Mortality | | | | | |
| JAG1 | Low | 38 | 7.6 (22.2) | 32 | 0.04 |
| | High | 6 | 55 (15.9) | 63 | |
| NOTCH 1 | Low | 32 | 34.9 (21.1) | 30.5 | 0.005 |
| | High | 5 | 70.2 (14.1) | 66 | |
| NOTCH 3 | Low | 40 | 37.8 (22.0) | 31.5 | 0.02 |
| | High | 6 | 53.8 (13.8) | 55.5 | |
| Relapse | | | | | |
| JAG1 | Low | 38 | 55.0 (19.7) | 51 | 0.06 |
| | High | 6 | 68.0 (9.7) | 71.5 | |
| NOTCH 1 | Low | 32 | 52.7 (18.4) | 52 | 0.004 |
| | High | 5 | 79.8 (11.3) | 74 | |
| NOTCH 3 | Low | 40 | 55.6 (19.3) | 52 | 0.09 |
| | High | 6 | 67.5 (10.5) | 71 | |

¹P-value from Mann-Whitney test comparing two groups

TABLE 6

| ID | Age | Pos Nodes | Size CA (cm) | Grade | ER | PR | Status | Survival (mo) | JAG1 | NOTCH 1 | NOTCH 3 |
|---|---|---|---|---|---|---|---|---|---|---|---|
| 1 | 55 | 0 | 1.1 | 1 | 1 | 0 | Alive | 148 | 2 | 2 | 1 |
| 2 | 72 | 0 | 2 | 2 | 0 | 0 | Deceased | 103 | 2 | 5 | 2 |
| 3 | 49 | 0 | 2 | 1 | 0 | 1 | Alive | 187 | 1 | 1 | 1 |
| 4 | 54 | 0 | 2 | 2 | 1 | 0 | Alive | 172 | 1 | 3 | 1 |
| 5 | 50 | 1 | 1.3 | 3 | | 0 | Deceased | 116 | 2 | 4 | 2 |
| 6 | 46 | 2 | 1.8 | 2 | | | Alive | 82 | | 1 | |
| 7 | 61 | 6 | 3.6 | 2 | 1 | 1 | Deceased | 34 | 2 | 3 | 2 |
| 8 | 33 | 7 | 1.8 | 2 | 0 | 0 | Alive | 217 | 2 | 2 | 1 |
| 9 | 54 | | 3.5 | 2 | 1 | 0 | Alive | 27 | | 4 | 1 |
| 10 | 76 | | 1.3 | 2 | 1 | 0 | Deceased | 0 | 3 | 5 | 1 |
| 11 | 75 | 0 | 2 | 2 | 1 | 1 | Alive | 3 | 1 | 4 | 1 |
| 12 | 48 | | 2.7 | 3 | 1 | 1 | Deceased | 12 | 2 | 2 | 1 |
| 13 | 68 | | 1.7 | 2 | 0 | 0 | Deceased | 23 | 1 | 2 | 8 |
| 14 | 46 | | 1.5 | 3 | 0 | 1 | Deceased | 11 | 1 | 2 | |
| 15 | 40 | | 0.7 | 3 | 0 | 0 | Deceased | 16 | 1 | 1 | |
| 16 | 81 | | 1 | 2 | 1 | 0 | Deceased | 57 | | | 1 |
| 17 | 45 | 2 | 1.2 | 2 | 1 | 0 | Alive | 110 | 3 | 5 | 1 |
| 18 | 49 | 1 | 1.3 | 2 | 0 | 1 | Alive | 120 | 4 | 4 | 2 |
| 19 | 41 | 29 | 4 | 2 | 1 | 0 | Alive | 102 | 3 | 4 | 1 |
| 20 | 76 | 7 | 1.3 | 2 | 1 | 1 | Deceased | 63 | 3 | 3 | 1 |
| 21 | 62 | 0 | 1.2 | 2 | 1 | 1 | Alive | 98 | 2 | 6 | 1 |
| 22 | 61 | 0 | 0.9 | 2 | 9 | 0 | Deceased | 103 | 10 | 3 | |
| 23 | 68 | 0 | 1 | 1 | 1 | 1 | Alive | 178 | | 8 | 1 |
| 24 | 63 | 0 | 0.7 | 2 | 1 | 0 | Deceased | 84 | 3 | 5 | 1 |
| 25 | 70 | 3 | 4 | 1 | 0 | 0 | Deceased | 56 | 1 | 1 | 1 |
| 26 | 74 | 2 | 5 | 2 | 1 | 0 | Deceased | 63 | 1 | 1 | 1 |
| 27 | 60 | 7 | 4 | 2 | 1 | 0 | Deceased | 83 | 1 | 6 | 1 |
| 28 | 51 | 2 | 5 | 2 | 0 | 1 | Deceased | 139 | 1 | 4 | 1 |
| 29 | 48 | 1 | 2 | 2 | 0 | 1 | Alive | 147 | 3 | | 1 |
| 30 | 51 | 2 | 3 | 2 | 0 | 0 | Alive | 39 | 2 | 2 | 1 |
| 31 | 55 | 1 | 1.8 | 2 | 1 | 1 | Deceased | 80 | 3 | 2 | 1 |
| 32 | 44 | 1 | 2 | 2 | 1 | 1 | Alive | 172 | 3 | 2 | 1 |
| 33 | 65 | 0 | 3.5 | 2 | 1 | 0 | Alive | 108 | 3 | 2 | 1 |
| 34 | 56 | 0 | 1.2 | 2 | 1 | 1 | Alive | 124 | | | 2 |
| 35 | 60 | 0 | 1.7 | 2 | 1 | 1 | Alive | 108 | 2 | 3 | 1 |
| 36 | 74 | 0 | 1.2 | 1 | 1 | 1 | Alive | 169 | | 3 | 1 |
| 37 | 47 | | 2.5 | 2 | 1 | 0 | Deceased | 30 | 1 | 2 | 1 |
| 38 | 71 | 9 | 4.3 | 1 | 1 | 0 | Deceased | 58 | 1 | 1 | 1 |
| 39 | 42 | | 1.7 | 2 | 0 | 0 | Deceased | 1 | 1 | 1 | 3 |
| 40 | 82 | | 2.2 | 2 | 1 | 1 | Deceased | 31 | 1 | 4 | 1 |
| 41 | 71 | 1 | 3 | 3 | 0 | 0 | Deceased | 69 | 4 | 8 | 7 |
| 42 | 58 | 4 | 2.5 | 2 | 1 | 0 | Alive | 110 | 2 | 5 | 2 |
| 43 | 39 | 15 | 2 | 1 | 0 | 0 | Alive | 119 | 3 | 3 | 1 |
| 44 | 49 | 10 | 0.9 | 1 | 0 | 1 | Alive | 66 | 3 | 4 | 3 |
| 45 | 48 | 0 | 0.9 | 2 | 1 | 0 | Deceased | 281 | 4 | 4 | 2 |
| 46 | 40 | 0 | 3.3 | 2 | 0 | 1 | Alive | 79 | 3 | 2 | 1 |
| 47 | 77 | 0 | 1.2 | 2 | 1 | 1 | Deceased | 85 | 4 | 3 | 1 |
| 48 | 75 | 0 | 1.5 | 2 | 1 | 1 | Alive | 103 | | | |
| 49 | 72 | | 1.9 | 2 | 1 | 0 | Deceased | 45 | 1 | 1 | 3 |
| 50 | 59 | 3 | 1 | 1 | 1 | 1 | Deceased | 131 | 2 | 3 | 2 |
| 51 | 65 | | 1.2 | 1 | 1 | 0 | Deceased | 22 | 1 | 1 | 1 |
| 52 | 51 | 29 | 1.8 | 2 | 0 | 0 | Deceased | 27 | 1 | 1 | 1 |
| 53 | 67 | 3 | 2.5 | 2 | 0 | 0 | Alive | 108 | | 2 | 1 |
| 54 | 42 | 1 | 3 | 3 | 1 | 0 | Deceased | 25 | | | 1 |
| 55 | 66 | 1 | 2 | 2 | 1 | 0 | Deceased | 53 | 4 | 5 | 1 |
| 56 | 72 | 1 | 3.7 | 3 | 0 | 0 | Deceased | 157 | 6 | 8 | 6 |
| 57 | 57 | 0 | 1.5 | 2 | 0 | 0 | Deceased | 28 | 6 | 2 | 1 |
| 58 | 41 | 0 | 4.1 | 3 | 0 | 0 | Deceased | 24 | | 8 | 4 |
| 59 | 71 | 0 | 0.8 | 2 | 1 | 0 | Alive | 68 | 4 | 3 | 1 |
| 60 | 67 | 0 | 2 | 3 | 0 | 0 | Deceased | 14 | 4 | 6 | 4 |
| 61 | 81 | 0 | 3 | 1 | | 1 | Deceased | 69 | | | |
| 62 | 73 | 0 | 4 | 2 | 1 | 1 | Alive | 133 | 1 | 1 | 3 |
| 63 | 45 | 0 | 0.9 | 2 | 1 | 1 | Alive | 142 | 1 | | 3 |
| 64 | 45 | 0 | 3 | 3 | 1 | 9 | Alive | 204 | 1 | 5 | 2 |
| 65 | 62 | 1 | 1.8 | 2 | 1 | 0 | Alive | 139 | | 1 | 1 |
| 66 | 81 | | 3 | 2 | 1 | 0 | Deceased | 11 | | 6 | 1 |
| 67 | 57 | 3 | 1.3 | 2 | 1 | 1 | Deceased | 19 | 3 | | 1 |
| 68 | 58 | | 1.5 | 2 | 1 | 0 | Deceased | 76 | 3 | 1 | 2 |
| 69 | 75 | 4 | 0.9 | 2 | 0 | | Deceased | 50 | 5 | 9 | 2 |
| 70 | 73 | 1 | 2 | 2 | 1 | 9 | Alive | 95 | 3 | 4 | 1 |
| 71 | 69 | 31 | 3 | 3 | 0 | 0 | Deceased | 147 | 4 | 3 | 1 |
| 72 | 29 | 4 | 1.4 | 1 | 1 | 0 | Deceased | 99 | 2 | 2 | 1 |
| 73 | 74 | 0 | 1.4 | 1 | 1 | 1 | Deceased | 189 | 1 | 3 | 1 |
| 74 | 78 | 0 | 0.8 | 1 | 1 | 0 | Alive | 110 | 1 | 2 | 1 |
| 75 | 33 | 0 | 2 | 3 | 0 | 0 | Alive | 127 | 1 | 3 | 2 |
| 76 | 70 | 0 | 0.8 | 1 | 1 | 9 | Deceased | 5 | 1 | | |
| 77 | 41 | 2 | 1.8 | 2 | | 0 | Deceased | 28 | | 8 | |
| 78 | 75 | 6 | 2 | 1 | 1 | 1 | Deceased | 57 | 6 | 4 | 1 |
| 79 | 75 | 9 | 1.2 | 1 | 1 | 0 | Deceased | 56 | 3 | 2 | 1 |
| 80 | 75 | 8 | 1.5 | 1 | 1 | 0 | Deceased | 77 | 3 | 4 | 4 |
| 81 | 48 | 1 | 5 | 2 | 0 | 0 | Deceased | 17 | 7 | 4 | 4 |
| 82 | 63 | 3 | 4.5 | 3 | 9 | 0 | Deceased | 23 | 3 | 2 | 1 |
| 83 | 71 | 4 | 2 | 2 | 1 | 0 | Deceased | 100 | 2 | 2 | 1 |
| 84 | 54 | 1 | 1.1 | 3 | 0 | 0 | Alive | 124 | 7 | 10 | 10 |
| 85 | 57 | 0 | 1 | 3 | 0 | 0 | Alive | 27 | 3 | 1 | 1 |
| 86 | 61 | 0 | 1 | 1 | 1 | 0 | Alive | 162 | 2 | 2 | 2 |
| 87 | 61 | 0 | 1.7 | 1 | 1 | 0 | Deceased | 238 | | | |
| 88 | 75 | 0 | 1.8 | 2 | 0 | 0 | Deceased | 46 | 1 | 3 | 2 |
| 89 | 47 | 2 | 3.6 | 2 | 0 | 0 | Deceased | 77 | 1 | 2 | 1 |
| 90 | 42 | 2 | 4 | 3 | 0 | 0 | Deceased | 21 | 3 | | 4 |
| 91 | 46 | 13 | 3 | 2 | 0 | 0 | Alive | 201 | 1 | 1 | 2 |
| 92 | 68 | 2 | 5 | 1 | 0 | 0 | Deceased | 54 | 1 | 1 | 1 |
| 93 | 63 | | 2 | 2 | 1 | 1 | Deceased | 12 | 5 | 6 | 2 |
| 94 | 71 | | 2.5 | 1 | 1 | 0 | Deceased | 2 | | 5 | 2 |
| 95 | 43 | | 1 | 2 | 1 | 1 | Deceased | 34 | 2 | 2 | 1 |
| 96 | 67 | | 1.4 | 2 | 1 | 1 | Deceased | 1 | 3 | 3 | 1 |
| 97 | 41 | 0 | 1.5 | 1 | 0 | 0 | Deceased | 108 | 1 | | |
| 98 | 64 | 0 | 2 | 3 | 0 | 0 | Alive | 94 | 1 | 10 | 4 |
| 99 | 45 | 0 | 3.5 | 2 | 1 | 1 | Alive | 129 | 1 | 2 | 2 |
| 100 | 50 | 0 | 1.4 | 3 | 0 | 0 | Alive | 41 | 2 | 3 | 2 |
| 101 | 45 | | 2.5 | 2 | 1 | 0 | Deceased | 34 | 5 | 7 | 1 |
| 102 | 73 | 11 | 3 | 1 | 1 | 1 | Deceased | 23 | 8 | 4 | 2 |
| 103 | 39 | | 1 | 2 | 0 | 0 | Deceased | 22 | 8 | 1 | 1 |
| 104 | 44 | | 1.5 | 2 | 9 | 1 | Deceased | 62 | | 1 | 1 |
| 105 | 65 | 1 | 2.2 | 1 | 1 | 0 | Alive | 100 | 6 | 6 | 3 |
| 106 | 68 | 31 | 3 | 2 | 1 | 9 | Alive | 104 | | 2 | 1 |
| 107 | 53 | 1 | 2.4 | 2 | 0 | 0 | Alive | 21 | 2 | 5 | 1 |
| 108 | 70 | 1 | 2.3 | 2 | 1 | 0 | Alive | 133 | | | 1 |
| 109 | 56 | 0 | 1.5 | 2 | 0 | 0 | Alive | 9 | 1 | 1 | 1 |
| 110 | 69 | 0 | 1.5 | 3 | 1 | 1 | Deceased | 155 | 1 | 3 | 1 |
| 111 | 53 | 0 | 2.3 | 3 | 1 | 0 | Alive | 25 | 1 | 7 | 2 |
| 112 | 76 | 0 | 2.5 | 2 | 1 | 1 | Deceased | 14 | 1 | 6 | 1 |
| 113 | 56 | | 3.5 | 2 | 0 | 0 | Deceased | 0 | 4 | 10 | 3 |
| 114 | 65 | 6 | 5 | 2 | 1 | 0 | Deceased | 43 | 10 | 8 | 1 |
| 115 | 77 | | 1.6 | 3 | 1 | 0 | Deceased | 1 | 8 | 6 | 2 |
| 116 | 54 | | 1.4 | 3 | 0 | 0 | Deceased | 7 | | | 1 |
| 117 | 60 | 9 | 3.5 | 3 | 1 | 0 | Deceased | 109 | 5 | 7 | 1 |
| 118 | 70 | 6 | 1.5 | 2 | 1 | 1 | Deceased | 22 | 5 | 5 | 1 |
| 119 | 63 | 4 | 3.5 | 3 | 0 | 0 | Alive | 101 | 4 | 9 | 1 |
| 120 | 66 | 2 | 2.5 | 1 | 1 | 9 | Alive | 133 | | 3 | 1 |
| 121 | 62 | 0 | 3.5 | 1 | 0 | 0 | Deceased | 42 | | | |
| 122 | 80 | 0 | 0.5 | 2 | 1 | 1 | Deceased | 102 | 4 | 4 | 1 |
| 123 | 61 | 0 | 2 | 2 | 0 | 0 | Deceased | 91 | 3 | 3 | 3 |
| 124 | 87 | 0 | 4 | 1 | 1 | 1 | Deceased | 77 | 3 | 7 | 3 |
| 125 | 66 | 1 | 1 | 2 | 1 | 0 | Alive | 98 | | 3 | 1 |
| 126 | 60 | 7 | 2.8 | 2 | 1 | 0 | Deceased | 122 | 3 | 4 | 1 |
| 127 | 55 | 1 | 2.7 | 1 | 1 | 1 | Deceased | 117 | | | |
| 128 | 43 | 1 | 1.5 | 3 | 0 | 0 | Deceased | 46 | 6 | 5 | 3 |
| 129 | 62 | | 6 | 2 | 1 | 1 | Deceased | 124 | | 2 | 1 |
| 130 | 39 | | 1.3 | 1 | 1 | 0 | Deceased | 20 | 2 | 1 | 1 |
| 131 | 58 | 0 | 1.3 | 2 | 1 | 0 | Alive | 11 | 2 | 2 | 1 |
| 132 | 47 | | | 2 | 0 | 0 | Deceased | 11 | 4 | 3 | 2 |
| 133 | 42 | 0 | 1 | 2 | 1 | 1 | Alive | 229 | | 2 | 1 |
| 134 | 76 | 0 | 0.7 | 2 | 1 | 0 | Deceased | 30 | 3 | 3 | 1 |
| 135 | 71 | 0 | 2.3 | 1 | 1 | 1 | Alive | 109 | 2 | 3 | 1 |
| 136 | 40 | 0 | 0.05 | 2 | 1 | 0 | Alive | 104 | 1 | | 1 |
| 137 | 44 | 1 | 2 | 2 | 0 | 0 | Alive | 127 | 9 | 2 | 1 |
| 138 | 56 | 1 | 1.7 | 2 | 0 | 0 | Deceased | 30 | 8 | 5 | 2 |
| 139 | 62 | 2 | 1.8 | 2 | 1 | | Alive | 120 | | 1 | 1 |
| 140 | 68 | 5 | 3.5 | 2 | 0 | 0 | Deceased | 80 | 3 | 3 | 1 |
| 141 | 85 | | 1.7 | 2 | 1 | 1 | Deceased | 5 | 2 | 2 | 1 |
| 142 | 47 | | 2 | 2 | 0 | 0 | Deceased | 11 | 1 | 1 | 2 |
| 143 | 70 | | 5 | 2 | 0 | 0 | Deceased | 1 | 1 | 1 | |
| 144 | 64 | 13 | 3.7 | 2 | 0 | 0 | Deceased | 19 | 3 | 1 | 1 |
| 145 | 44 | 9 | 4 | 2 | 1 | 1 | Deceased | 49 | 1 | 1 | 1 |
| 146 | 36 | 7 | 4 | 2 | 1 | 1 | Deceased | 51 | | 1 | 1 |
| 147 | 51 | 2 | 0.9 | 1 | 1 | 1 | Alive | 115 | 1 | 2 | 1 |
| 148 | 65 | 5 | 3.7 | 2 | 1 | | Alive | 115 | | | 2 |
| 149 | 41 | 12 | 2.5 | 2 | 1 | 1 | Deceased | 57 | 4 | 2 | 1 |
| 150 | 89 | 1 | 2 | 2 | 1 | 1 | Deceased | 4 | | | 1 |
| 151 | 45 | 10 | 5 | 3 | 0 | 0 | Deceased | 12 | 5 | 6 | 5 |
| 152 | 76 | | 2 | 2 | 1 | | Alive | 15 | 6 | 3 | 4 |
| 153 | 52 | 0 | 3 | 2 | 1 | 0 | Alive | 237 | 3 | 2 | 1 |
| 154 | 77 | 0 | 1 | 1 | 1 | 0 | Alive | 150 | 2 | 1 | 1 |
| 155 | 70 | 0 | 0.6 | 2 | 1 | 1 | Alive | 95 | 3 | 1 | 2 |
| 156 | 65 | 0 | 2.2 | 1 | 1 | 0 | Alive | 159 | | 1 | 1 |
| 157 | 42 | 1 | 2.3 | 3 | 0 | 0 | Alive | 100 | 1 | 3 | 2 |
| 158 | 69 | 15 | 1.4 | 2 | 1 | 1 | Deceased | 72 | 2 | 2 | 1 |
| 159 | 33 | 2 | 4 | 3 | 0 | 0 | Deceased | 32 | 2 | 1 | 1 |
| 160 | 47 | 3 | 1 | 2 | 0 | 0 | Alive | 75 | 2 | 2 | |
| 161 | 46 | 1 | 5 | 2 | 1 | | Deceased | 36 | 5 | 8 | 3 |
| 162 | 44 | | 0.8 | 3 | 0 | 0 | Deceased | 12 | 4 | 1 | 2 |
| 163 | 64 | | 3.5 | 2 | 1 | 0 | Deceased | 71 | 9 | 2 | 1 |
| 164 | 72 | | 2.5 | 2 | 1 | | Deceased | 11 | 5 | 1 | 1 |
| 165 | 43 | 0 | 1.5 | 2 | 1 | 0 | Alive | 192 | 3 | 5 | 2 |
| 166 | 73 | 0 | 0.6 | 2 | 1 | 1 | Alive | 74 | 3 | 1 | 1 |
| 167 | 70 | 0 | 2.7 | 2 | 1 | 1 | Alive | 47 | 3 | 1 | 1 |
| 168 | 62 | 0 | 0.7 | 3 | 0 | 0 | Alive | 195 | 2 | 1 | 2 |
| 169 | 46 | 2 | 2 | 3 | 0 | 0 | Deceased | 75 | 3 | 8 | 1 |
| 170 | 40 | 23 | 2 | 2 | 0 | 0 | Deceased | 14 | 2 | 1 | 2 |
| 171 | 41 | 30 | 1.5 | 2 | 0 | 0 | Deceased | 35 | 6 | 2 | 2 |
| 172 | 62 | 3 | 3.7 | 3 | 0 | 0 | Alive | 1 | 2 | 2 | 2 |
| 173 | 56 | | 3 | 2 | 1 | 0 | Deceased | 8 | 2 | 6 | 2 |
| 174 | 78 | | 2 | 2 | 1 | 0 | Deceased | 15 | 3 | 4 | 1 |
| 175 | 66 | | 4.5 | 2 | 1 | 0 | Deceased | 38 | 3 | 1 | 1 |
| 176 | 67 | 0 | 1.5 | 2 | 1 | 1 | Deceased | 208 | 2 | 2 | 1 |
| 177 | 68 | 0 | 0.9 | 1 | 1 | 1 | Deceased | 80 | 3 | 2 | 1 |
| 178 | 42 | 0 | 3.5 | 2 | 0 | 0 | Deceased | 19 | 8 | 7 | 3 |
| 179 | 44 | 0 | 3.8 | 3 | 0 | 0 | Alive | 79 | 4 | 2 | 4 |
| 180 | 62 | 0 | 0.9 | 2 | 1 | 1 | Deceased | 82 | 1 | 1 | 1 |
| 181 | 45 | 16 | 3 | 3 | 0 | 1 | Deceased | 44 | 3 | 3 | 1 |
| 182 | 49 | 18 | 4.7 | 2 | 1 | 1 | Alive | 16 | 2 | 3 | 1 |
| 183 | 53 | 10 | 5 | 1 | 1 | 1 | Deceased | 62 | 3 | 2 | 1 |
| 184 | 61 | 4 | 2.5 | 2 | 1 | 1 | Deceased | 52 | 2 | 1 | 1 |
| 185 | 74 | | 2.5 | 2 | 1 | 0 | Deceased | 11 | 4 | 7 | 4 |
| 186 | 68 | | 1.5 | 2 | 1 | 0 | Deceased | 26 | 2 | | 1 |
| 187 | 80 | 10 | 4.5 | 2 | 1 | 0 | Deceased | 81 | 3 | 6 | 1 |
| 188 | 43 | 1 | 3.5 | 2 | 1 | 0 | Deceased | 78 | 5 | 7 | 1 |
| 189 | 60 | 0 | 1.5 | 2 | 1 | 0 | Alive | 130 | 3 | 3 | |
| 190 | 75 | 0 | 1 | 1 | 1 | 0 | Alive | 83 | 4 | 3 | 1 |
| 191 | 61 | 0 | 0.8 | 1 | 1 | 1 | Alive | 95 | 3 | | |
| 192 | 67 | 0 | 1.3 | 1 | 1 | 0 | Alive | 77 | 1 | | 1 |

TABLE 7a
NCI Survival Analysis - Cox Proportional Hazards Model

| | | HR (95% CI) | | | |
|---|---|---|---|---|---|
| | N | per category | IQR¹ | HR over IQR² | P-value³ |
| JAG1 | 161 | 1.11 (1.02-1.21) | 3 | 1.37 | 0.02 |
| NOTCH 1 | 170 | 1.06 (0.97-1.15) | 3 | 1.18 | 0.21 |
| NOTCH 3 | 176 | 1.06 (0.94-1.20) | 1 | 1.06 | 0.36 |

¹IQR (interquartile range) = Third quartile value − first quartile value
²Hazard ratio comparing a subject in the highest quarter for the measure to a subject in the lowest quarter
³P-value from Cox proportional hazards model

TABLE 7b
NCI Survival Analysis - Comparison of tumors expressing high
(Hi) and low (Lo) levels of JAG1, NOTCH 1 and NOTCH 3 mRNA

| | | | 5 year | Median | |
|---|---|---|---|---|---|
| | | | survival | survival time | P- |
| | | N | (95% CI) | (95% CI) | value¹ |
| JAG1 | Lo | 117 | 65% (56-74) | 83 mo (76-122) | 0.01 |
| | Hi | 44 | 42% (27-57) | 50 mo (30-78) | |
| NOTCH 1 | Lo | 126 | 64% (55-72) | 91 mo (72-131) | 0.02 |
| | Hi | 44 | 49% (34-64) | 53 mo (30-83) | |
| NOTCH 3 | Lo | 150 | 61% (53-69) | 82 mo (71-122) | 0.08 |
| | Hi | 26 | 48% (29-68) | 46 mo (23-91) | |
| JAG1^{HI}/N1^{Hi} | | 22 | 32% (12-51) | 40 mo (19-69) | 0.003 |
| JAG1^{HI}/N1^{Hi} excluded | | 129 | 63% (54-71) | 81 mo (72-103) | |
| Combined | J1^{Lo}/N1^{Lo} | 90 | 62% (51-72) | 80 mo (63-131) | |
| JAG1/N1 | J1^{Lo}/N1^{Hi} | 17 | 82% | 84 mo (77-NR²) | 0.02 |
| | | | (64-100) | | |
| | J1^{Hi}/N1^{Lo} | 22 | 53% (31-74) | 71 mo (28-103) | |
| | J1^{Hi}/N1^{Hi} | 22 | 32% (12-51) | 40 mo (19-69) | |

¹P-value from log rank test
²Upper limit not reached

TABLE 8
BI-VARIATE SURVIVAL MODELS FOR JAG1 INDIVIDUALLY
CONTROLLING FOR COMMON RISK FACTORS

| | TOTAL N | OR (95% CI) per unit | P-VALUE¹ |
|---|---|---|---|
| Metastases | 160 | 5.32 (3.52-8.05) | <0.001 |
| JAG1 | | 1.09 (1.01-1.19) | 0.04 |
| Age | 161 | 1.02 (1.01-1.03) | 0.04 |
| JAG1 | | 1.11 (1.01-1.21) | 0.03 |
| Tumour size | 160 | 1.23 (1.05-1.44) | 0.01 |
| JAG1 | | 1.10 (1.01-1.21) | 0.03 |
| Node Positive | 129 | 2.54 (1.49-4.36) | <0.001 |
| JAG1 | | 1.12 (1.01-1.25) | 0.05 |
| ER Positive | 158 | 0.89 (0.59-1.35) | 0.59 |
| JAG1 | | 1.12 (1.02-1.24) | 0.02 |
| Grade² | 161 | | 0.10 |
| I | | 1.00 | |
| II | | 1.39 (0.81-2.40) | |
| III | | 1.29 (0.67-2.49) | |
| JAG1 | | 1.10 (1.01-1.20) | 0.04 |

¹P-value from Cox proportional hazards model
²Grade I is reference category

TABLE 9
JAG1, NOTCH1 and NOTCH3 co-expression
High is defined as top quartile; low is the lower three quartiles
Percentages given in table are out of the total

JAG1

| | | Low | High | Total |
|---|---|---|---|---|
| NOTCH1 | Low | 90 (60%) | 22 (15%) | 112 |
| | High | 17 (11%) | 22 (15%) | 39 |
| | Total | 107 | 44 | 151 |

Chi-squared test: p = 0.001. If JAG1 and NOTCH1 were
independent of each other we would expect only 11 (7.5%)
subjects in the high-high category.

JAG1

| | | Low | High | Total |
|---|---|---|---|---|
| NOTCH3 | Low | 98 (64%) | 29 (19%) | 127 |
| | High | 11 (7%) | 14 (9%) | 25 |
| | Total | 109 | 43 | 152 |

Chi-squared test: p = 0.001. If JAG1 and NOTCH3 were
independent of each other we would expect only 7 (5%) subjects
in the high-high category.

NOTCH3

| | | Low | High | Total |
|---|---|---|---|---|
| NOTCH1 | Low | 109 (67%) | 10 (6%) | 119 |
| | High | 29 (18%) | 14 (9%) | 43 |
| | Total | 138 | 24 | 162 |

Chi-squared test: p = 0.001. If NOTCH1 and NOTCH3 were
independent of each other we would expect only 6 (4%) subjects
in the high-high category.

TABLE 10

| Jagged 1 | | | 5 Year Survival | Median Survival |
|---|---|---|---|---|
| Allred | N | Dead | (95% CI) | (95% CI) |
| Bottom 3 quartiles | 126 | 73 | 64% (55-72) | 91 mo (77-124) |
| (0-4) | | | | |
| Top quartile | 26 | 21 | 40% (21-58) | 43 mo (19-71) |
| (5-7) | | | | |

Log rank test: p < 0.001

TABLE 11

| Notch 1 | | | 5 Year Survival | Median Survival |
|---|---|---|---|---|
| Allred | N | Dead | (95% CI) | (95% CI) |
| Bottom 3 quartiles | 157 | 92 | 61% (52-68) | 82 mo (69-116) |
| (0-7) | | | | |
| 4^{th} quartile | 13 | 11 | 46% (19-70) | 50 mo (19-109) |
| (8) | | | | |

Log rank test: p = 0.08

1. A method for assessing prognosis for a subject having a breast tumor, comprising determining the level of expression of at least one Notch receptor gene, Notch ligand gene or Notch signaling target gene in the tumor.

2. The method of claim 1 comprising determining the level of expression of at least one gene selected from the group consisting of Notch 1. Notch 3, Jagged 1, Hey 1, Hey 2, HevL, Hes 1, Hes 2, Hes 3, Hes 4. Hes 5, Hes 6 and Hes 7.

3. The method of claim 1 wherein an increased level of expression of the at least one gene compared to the level of expression of the at least one gene in normal tissue is indicative of a poor prognosis for the subject.

4. The method of any one of claims 1 wherein the subject is a human subject.

5. The method of claim 4 wherein the at least one gene is selected from the group consisting of NOTCH 1, NOTCH 3 and JAG 1.

6. The method of claim 4 wherein the levels of expression of NOTCH I and JAG 1 are determined.

7. The method of claim 4 wherein the levels of expression of NOTCH 3 and JAG 1 are determined.

8. The method of claim 4 wherein the levels of expression of NOTCH 1 and NOTCH 3 are determined.

9-11. (canceled)

12. The method of any one of claims 1 wherein the level of gene expression is determined by a nucleic-acid based assay or by an expressed protein-based assay.

13-15. (canceled)

16. A method of diagnosing breast cancer in a subject comprising determining the level of expression of at least one Notch receptor gene, Notch ligand gene or Notch signaling target gene in a breast tissue sample obtained from the subject.

17. The method of claim 16 comprising determining the level of expression of at least one gene selected from the group consisting of Notch 1, Notch 3, Jagged 1, Hey 1, Hey 2, HeyL, Hes 1, Hes 2, Hes 3, Hes 4, Hes 5, Hes 6 and Hes 7.

18. The method of claim 16 wherein an increased level of expression of the at least one gene compared to the level of expression of the at least one gene in normal breast tissue is indicative of breast cancer in the subject.

19. The method of any one of claims 16 wherein the subject is a human subject.

20. The method of claim 19 wherein the at least one gene is selected from the group consisting of NOTCH 1, NOTCH 3 and JAG 1.

21. The method of claim 19 wherein the levels of expression of NOTCH 1 and JAG 1 are determined.

22. The method of claim 19 wherein the levels of expression of NOTCH 3 and JAG 1 are determined.

23. The method of claim 19 wherein the levels of expression of NOTCH 1 and NOTCH 3 are determined.

24-26. (canceled)

27. The method of any one of claims 16 wherein the level of gene expression is determined by a nucleic-acid based assay or by an expressed protein-based assay.

28-30. (canceled)

31. A method for treating a subject suffering from a breast tumor associated with increased Notch signaling comprising administering to the subject an effective amount of an inhibitor of Notch signaling.

32. The method of claim 31 wherein the inhibitor of Notch signaling reduces expression of at least one gene selected from the group consisting of Notch 1, Notch 3, Jagged 1. Hey 1, Hey 2, HevL and Hes 1 to Hes 7.

33. The method of claim 31 wherein the inhibitor of Notch signaling reduces the activity of at least one protein selected from the group consisting of Notch 1, Notch 3, Jagged 1, Hey 1, Hey 2, HeyL and Hes 1 to Hes 7.

34. The method of claim 32 wherein the inhibitor of Notch signaling is one or more antisense oligonucleotides or siRNAs which hybridise to the at least one gene or a complementary sequence thereof.

35. The method of claim 33 wherein the inhibitor of Notch signaling is a γ˜secretase inhibitor.

36. The method of any one of claims 31 wherein the subject is human and wherein the inhibitor of Notch signaling reduces expression of at least one of NOTCH 1, NOTCH 3 and JAG 1.

37-48. (canceled)

49. A method for screening a candidate compound for its potential usefulness in the treatment of breast cancer comprising contacting a cell or cells with the candidate compound under conditions which permit expression in the cell(s) of at least one gene selected from the group consisting of Notch I, Notch 3, Jagged 1 Hey 1, Hey 2, HevL and Hes 1 to Hes 7 and determining the level of expression of said at least one gene in the cell(s), wherein a lower level of expression compared to the level of expression in the cell or cells in the absence of the compound indicates the potential usefulness of the compound in the treatment of breast cancer.
